(19) United States
(12) Patent Application Publication
(10) Pub. No.: US 2013/0165489 A1
(43) Pub. Date: Jun. 27, 2013

(54) Small Molecule Modulators of HIV-1 Capsid Stability and Methods Thereof

(71) Applicants: Simon Cocklin, Philadelphia, PA (US); Sandhya Kortagere, Newtown, PA (US); Amos B. Smith, III, Merion, PA (US)

(72) Inventors: Simon Cocklin, Philadelphia, PA (US); Sandhya Kortagere, Newtown, PA (US); Amos B. Smith, III, Merion, PA (US)

(73) Assignees: The Trustees of the University of Pennsylvania, Philadelphia, PA (US); Philadelphia Health & Education Corporation d/b/a Drexel University College of Medicine, Philadelphia, PA (US)

(21) Appl. No.: 13/643,392

(22) PCT Filed: Apr. 25, 2011
(86) PCT No.: PCT/US11/33789

§ 371 Date: Mar. 15, 2013

Related U.S. Application Data
(60) Provisional application No. 61/330,748, filed on May 3, 2010.

(51) Int. Cl. C07D 417/12; C07D 405/10; A61K 31/4164; A61K 45/06; A61K 31/4178; A61K 31/427; C07D 405/14; C07D 233/64

(52) U.S. Cl. CPC C07D 417/12; C07D 405/14; C07D 405/10; C07D 233/64; A61K 45/06; A61K 31/4178; A61K 31/427; A61K 31/4164
USPC 514/372; 548/343.5; 514/400; 548/311.4; 514/397

(57) ABSTRACT

The present invention includes a method of inhibiting, suppressing or preventing a viral infection in a subject, comprising administering to the subject a pharmaceutical composition comprising one or more of the compounds useful within the invention.

### BACKGROUND OF THE INVENTION

Human immunodeficiency virus type 1 (HIV-1), the major causative agent of acquired immunodeficiency syndrome (AIDS), is a retrovirus of the genus Lentivirinae. Retroviruses are small enveloped viruses that contain a diploid RNA genome. Each HIV-1 viral particle is composed of three discrete layers. The external surface of the virus is comprised of a lipid bilayer that is derived from the infected host cell. Embedded within this membrane are the viral envelope glycoproteins. The viral glycoproteins are organized on the virion surface as trimeric spikes, composed of three gp120 molecules non-covalently linked to three gp41 molecules, and function to mediate the entry of HIV-1 into susceptible cells. Below the lipid bilayer is a layer formed of the N-terminal region of the Gag polyprotein, known as the matrix (MA) protein. The third layer of the viral particle serves to protect the viral genome and replicative enzymes of HIV-1. This layer is a shell consisting of assembled mature capsid (CA) protein.

The HIV-1 CA protein (SEQ ID NO:1) performs essential roles both early and late in the life cycle of HIV: one structural, in which it forms a protein shell that shields both the viral genome and the replicative enzymes of HIV-1, and the other regulatory, in which the precise temporal disassembly of this shell coordinates post-entry events such as reverse transcription.

The HIV-1 CA protein is initially translated as the central region of the Gag polyprotein, where it functions in viral assembly and in packaging the cellular protein prolyl isomerase, cyclophilin A (CypA). As the virus buds, Gag is processed by the viral protease to produce three discrete new proteins—MA protein, CA protein, and nucleocapsid (NC)—as well as several smaller spacer peptides. After HIV-1 CA protein has been liberated by proteolytic processing, it rearranges into the conical core structure that surrounds the viral genome at the center of the mature virus.

The HIV-1 capsid shell is composed of about 250 CA protein hexamers and 12 CA protein pentamers, comprising about 1,500 monomeric CA proteins in all. The multimers interact non-covalently to form the shell's curved surface. CA protein itself is composed of two domains: the N-terminal domain (CA_{NTD}) and the C-terminal domain (CA_{CTD}). Both of these domains make critical inter- and intradomain interactions that are critical for the formation of the capsid shell. The structures of the individual domains, the NTD hexamer, the single CA protein, and the CA_{NTD} linked to MA have been determined (Gamble et al., 1996, Cell 87(7):1285-1294; Ganser-Pornillos et al., 2007, Cell 131(1):70-79; Ganser-Pornillos et al., 2008, Curr. Opin. Struct. Biol. 18(2):203-217; Gitti et al., 1996, Science 273(5272):231-235; Kelly et al., 2006, Biochemistry 45(38):11257-11266; Kelly et al., 2007, J. Mol. Biol. 373(2):355-366; Worthylake et al., 1999, Acta Crystallogr. D Biol. Crystallogr. 55(Pt 1):85-92). Both CA_{NTD} and CA_{CTD} are predominantly helical and are connected by a short flexible linker.

The CA_{NTD} is composed of an N-terminal β-hairpin, seven α-helices, and an extended loop connecting helices 4 and 5 that binds CypA. CA protein residues 146 and 147 act as a flexible linker that connects the CA_{NTD} with the smaller CA_{CTD}, which is composed of four α-helices. The CTD dimerizes in solution and in the crystal, and contains an essential stretch of 20 amino acids (the major homology region) that is highly conserved in all retroviruses.

Recently, researchers, guided by previous electron cryomicroscopy and modeling studies, engineered the HIV-1 CA protein to be stable, soluble, and amenable to crystallization (Pornillos et al., Cell 2009; 137(7):1282-1292), and determined the structure of the CA protein hexamer to a resolution of 2 Å. The structures reveal that six NTDs form the rigid core of hexameric CA protein, and six CTDs form the hexamer's much more flexible outer ring. Dimeric interactions between CTDs of neighboring hexamers hold the capsid together.

NTD-NTD interactions are responsible for the formation of the HIV-1 CA protein hexameric configuration. NTD-NTD interactions are mediated through helices 1, 2, and 3, which associate as an 18-helix bundle in the center of the hexamer. The interface is primarily stabilized by hydrophilic contacts (bridging water molecules, hydrogen bonds, and salt bridges). However, the interface contains a small hydrophobic core of residues (L20, P38, M39, A42, and T58) (Pornillos et al., 2009, Cell 137(7):1282-1292).

Extensive mutagenesis of the NTD domain has been performed. In addition to the mutations that perturb normal particle assembly, specific mutations in the NTD that either destabilized or stabilized the structure of the CA protein hexamer had adverse affects on viral replication (Ganser-Pornillos et al., 2004, J. Virol. 78(5):2545-2552; von Schwedler et al., 203, J. Virol. 77(9):5439-5450; Forshey et al., 2002, J. Virol. 76(11):5667-5677). Some residues of importance include the intersubunit stabilizing residues (E45, E128, and R132), the intersubunit destabilizing residues (R18, N21, P38, Q63, Q67, and L136), and the residues that when mutated reduce the rate of polymerization (A22, E28, and E29). Perhaps most interesting are two residues, M39 and A42, that when mutated completely prevent capsid assembly, as these may denote a potential “hotspot” for hexamerization. All of these types of mutations (stabilizing, destabilizing, and polymerization rate reducing) have a detrimental effect on the fitness of the virus. The inhibitory effects of mutations that modulate the stability of the capsid further highlight the need for a very delicate balance of favorable and unfavorable interactions within HIV-1 CA protein to allow assembly but also facilitate the uncoating process following infection.

CA-targeted small-molecule drugs have not yet been developed. Two inhibitors have been found to impede in vitro capsid assembly: the small-molecule inhibitor CAP-1 [(N-(3-chloro-4-methylphenyl)-N′-[2-[([5-[(dimethylamino)-methyl]-2-furyl]-methyl)-sulfanyl]ethyl]urea] and the peptide CA-I. CAP-1 binds to the CA_{NTD} with an equilibrium dissociation constant (K_{D}) of ˜0.8 mM, while CA-I binds to CA_{CTD} with a K_{D} of ˜15 μM (Tang et al., 2003, J. Mol. Biol. 327(5):1013-1020; Ternois et al., 2005, Nat. Struct. Mol. Biol. 12(8):678-682). Neither of these inhibitors disrupts HIV-1 replication in vivo. However, a modified CA-I peptide, termed NYAD-1, has recently been shown to penetrate cells and inhibit a broad spectrum of HIV-1 subtypes (Zhang et al., 2008, J. Mol. Biol. 378(3):565-580). The inhibitors CAP-1, CA-I, and NYAD-1 bind to different domains of CA protein but may work in a similar manner. Analysis of the binding site of CAP-1 within the structure of the hexameric complex confirms that it nestles into a hidden pocket in the NTD adjacent to the NTD-CTD interface (Kelly et al., 2007, J. Mol. Biol. 373(2):355-366; Pornillos et al., 2009, Cell 137(7):1282-1292). CAP-1 is proposed to function by altering the local geometry required to make the NTD-CTD interface. However, the CA-I and NYAD-1 peptides bind to a conserved hydrophobic cleft in the CTD (Ternois et al., 2005, Nat. Struct. Mol. Biol. 12(8):678-682). Inspection of the CA-1/NYAD-1 peptide binding site in the context of the hexamer points to two potential mechanisms of action: the direct disruption of the NTD-CTD interaction or the induction of non-productive capsid conformation (Pornillos et al., 2009, Cell 137(7):1282-1292).

More recently, studies completed by Pfizer have identified a novel compound through high throughput screening, PF-3450074 (PF74), which targets CA and inhibits HIV-1 at an early stage affecting proper reverse transcription (Blair et al., 2010, PLoS Pathog 6:e1001220; Shi, J., et al., 2010, J Virol 85(1):542-49). The compound destabilized the capsid and exerted antiviral effects by triggering a premature uncoating of HIV-1, mimicking the action of retrovirus restriction factor TRIM5α (tripartite motif-containing protein 5) (Shi, J., et al., 2011, J. Virol. 85(1):542-49). The binding site of PF74 was determined by X-ray crystallography and is situated in the NTD of the CA protein, comprising a preformed pocket in HIV-1 CA bounded by helices 3, 4, 5 and 7 and involving interactions with residues Asn-53, Leu-56, Val-59, Gln-63, Met-66, Gln-67, Leu-69, Lys-70, Ile-73, Ala-105, Thr-107, Tyr-130 (Blair et al., 2010, PLoS Pathog 6:e1001220). This study has highlighted the NTD of HIV-1 CA and the process of uncoating as viable targets in HIV-1 replication.

There are many other types of viruses that pose health risks to animals, and to human beings in special. Dengue fever (DF), dengue hemorrhagic fever (DHF), and dengue shock syndrome (DSS) are caused by four closely related serotypes of the Dengue virus (DENV), a mosquito-borne flavivirus. DF manifests as a sudden onset of severe headache, muscle and joint pains, fever, and rash. The dengue rash is characteristically bright red petechiae and usually appears first on the lower limbs and the chest; in some patients, it spreads to cover most of the body. DHF is a potentially lethal complication, characterized by fever, abdominal pain, vomiting, and bleeding, that mainly affects children. Worldwide, dengue currently infects between 50 and 100 million people a year, killing an estimated 25,000, many of whom are children. The global incidence of dengue has grown dramatically in recent decades, with approximately two-fifths of the world's population now at risk. Although dengue is predominantly found in tropical and subtropical climates, reported cases along the Texas-Mexico border and extremely recently in Key West, Miami Beach, and Ocala, Fla., have raised concerns about the potential for reemergence of dengue in the continental United States.

Currently, no vaccine or specific antiviral treatments are available for dengue. Development of an effective vaccine is stymied by a number of obstacles including the existence of 4 types of DENV (serotypes 1-4); the fact that antibodies developed against one subtype protect only against that subtype; and the fact that antibodies raised against one serotype of DENV may actually assist in infection by another serotype. In the absence of a viable vaccine, the pursuit of prophylactic intervention is the next logical step. However, chemical compounds for the treatment of DENV would also have to be active against all 4 serotypes of DENV to be effective.

West Nile virus is an emerging human pathogen for which specific antiviral therapy has not been developed. Over the past decade, WNV has spread rapidly via mosquito transmission from infected migratory birds to humans. It is estimated that about 20% of people who become infected with WNV will develop West Nile fever. Symptoms include fever, headache, tiredness, and body aches, occasionally with a skin rash (on the trunk of the body) and swollen lymph glands. The symptoms of severe disease (also called neuroinvasive disease, such as West Nile encephalitis or meningitis or West Nile poliomyelitis) include headache, high fever, neck stiffness, stupor, disorientation, coma, tremors, convulsions, muscle weakness, and paralysis. Although severe illness is relatively rare, it is estimated that approximately 1 in 150 persons infected with WNV will develop a more severe form of disease. Serious illness can occur in people of any age; however, people over age 50 and some immune-compromised persons are at the highest risk for severe illness when infected with WNV.

Respiratory syncytial virus (RSV) is a leading cause of pneumonia and bronchiolitis in infants and young children and an important pathogen in elderly and immune suppressed persons. The only intervention currently available is a monoclonal antibody against the RSV fusion protein, which has shown utility as a prophylactic for high-risk premature infants, but which has not shown post-infection therapeutic efficacy in the specific RSV-infected populations studied. Therefore, for the major susceptible populations, a great need for effective treatment remains.

The HIV-1 CA protein thus plays both structural and regulatory roles in the life cycle of HIV-1. There remains a need in the art to identify novel small-molecule inhibitors that bind to HIV-1 CA protein and interfere with one or more of its biological functions, leading to impairment of HIV-1 life cycle and infection. There also remains a need in the art to identify novel small-molecule inhibitors that prevent or treat viral infections caused by viruses such as dengue fever, dengue hemorrhagic fever, dengue shock syndrome, West Nile virus infection, and respiratory syncytial virus infection. The present invention fulfills these needs.

### BRIEF SUMMARY OF THE INVENTION

The invention includes a composition comprising a compound of Formula (III):

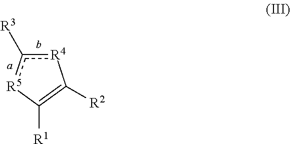

wherein in Formula (III):

R¹, R² and R³ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl, and

R⁴ and R⁵ are such that:
(i) if ‘a’ is a double bond and ‘b’ is a single bond, then R⁵ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or
(ii) if ‘a’ is a single bond and ‘b’ is a double bond, then R⁵ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂;
   or a salt thereof.

In one embodiment, in Formula (III) R⁴ and R⁵ are such that:

(i) if ‘a’ is a double bond and ‘b’ is a single bond, then R⁵ is N or CH, and R⁴ is NH or N-alkyl, or

(ii) if ‘a’ is a single bond and ‘b’ is a double bond, then R⁵ is NH or N-alkyl, and R⁴ is N or CH.

In one embodiment, in Formula (III) R⁴ and R⁵ are such that:

(i) if ‘a’ is a double bond and ‘b’ is a single bond, then R⁵ is N, and R⁴ is NH or N-alkyl, or

(ii) if ‘a’ is a single bond and ‘b’ is a double bond, then R⁵ is NH or N-alkyl, and R⁴ is N.

In one embodiment, the compound is 4-(4,5-diphenyl-1H-imidazol-2-yl)benzoic acid (CMPD-E) or a salt thereof. In another embodiment, the composition further comprises a pharmaceutically acceptable carrier.

The invention also includes a compound of Formula (Ib):

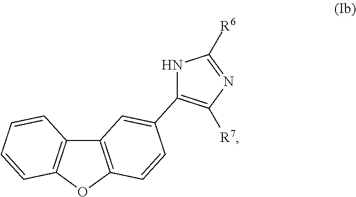

wherein in Formula (Ib) R⁶ and R⁷ are independently alkyl, halo alkyl, substituted alkyl, alkoxy, aryl, substituted aryl, —SO₂NH₂, —SO₂NH-alkyl, —SO₂NH-substituted alkyl—SO₂NH-aryl, —SO₂NH-substituted aryl, heteroaryl, substituted heteroaryl, alkoxycarbonyl, alkylthio, nitromethyl, or 2-nitroethyl, or a salt thereof.

In one embodiment, the compound is 4-(5-(dibenzo[b,d]furan-2-yl)-4-phenyl-1H-imidazol-2-yl)benzoic acid (CMPD-C) or a salt thereof. In another embodiment, the composition further comprises a pharmaceutically acceptable carrier.

The invention further includes a method of inhibiting, suppressing or preventing an HIV-1 infection in a subject in need thereof. The method comprises administering to the subject a composition comprising a therapeutically effective amount of at least one compound selected from the group consisting of:

(a) a compound of Formula (I):

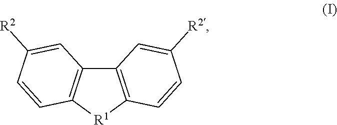

wherein in Formula (I):

R¹ is O, S, NH, N-alkyl, N—C(O)NH₂, N—CH₂C(O)NH₂, N—CH₂CH₂C(O)NH₂, CH₂, CH-alkyl, CH-OMe, CH-OEt, CH—C(O)NH₂, CH—CH₂C(O)NH₂, or CH—CH₂CH₂C(O)NH₂;

R² and R^{2′} are independently H or

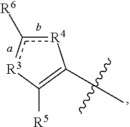

wherein,
(i) if ‘a’ is a double bond and ‘b’ is a single bond, then R³ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or
(ii) if ‘a’ is a single bond and ‘b’ is a double bond, then R³ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂; with the proviso that if R² is H then R^{2′} is not H; and

R⁵ and R⁶ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl;

(b) a compound of Formula (II):

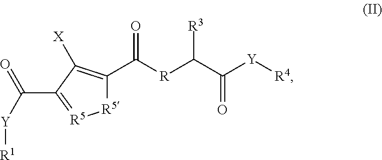

wherein:

R is NR₂, CHR₂, O or S;

R¹, R², R³ and R⁴ are independently H, alkyl, substituted alkyl, cycloalkyl, substituted cycloalkyl, aryl, substituted aryl, benzyl, substituted benzyl, heteroaryl, or substituted heteroaryl;

R⁵ is N or CH;

R^{5′} is CH₂, NH, S or O;

X is —NH₂, —NHR¹, —NR¹R², —OH, cyano, alkyl, alkoxy, halogen, sulfonamide, aryl, substituted aryl, heteroaryl or substituted heteroaryl; and,

each occurrence of Y is independently NH, NR¹, O, CH₂, CHR¹ or CR¹R²;

(c) a compound of Formula (III):

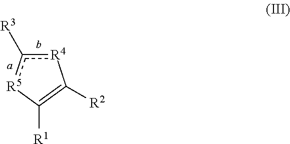

wherein in Formula (III):

R¹, R² and R³ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl,

R⁴ and R⁵ are such that:

(i) if ‘a’ is a double bond and ‘b’ is a single bond, then R⁵ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or

(ii) if ‘a’ is a single bond and ‘b’ is a double bond, then R⁵ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂; a mixture thereof and a pharmaceutically acceptable salt thereof.

In one embodiment, the compound of Formula (I) is a compound of Formula (Ia):

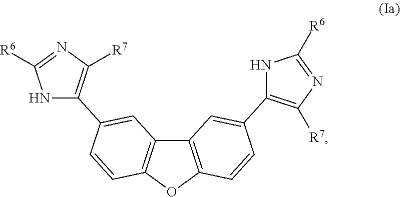

wherein in Formula (Ia):

R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

In one embodiment, the compound of Formula (I) is a compound of Formula (Ib):

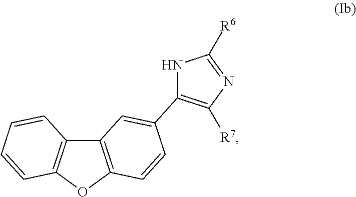

wherein in Formula (Ib):

R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

In one embodiment, in the compound of Formula (III) R⁴ and R⁵ are such that:

(i) if ‘a’ is a double bond and ‘b’ is a single bond, then R⁵ is N, and R⁴ is NH or N-alkyl, or

(ii) if ‘a’ is a single bond and ‘b’ is a double bond, then R⁵ is NH or N-alkyl, and R⁴ is N.

In one embodiment, the compound is selected from the group consisting of 4,4′-(5,5′-(dibenzo[b,d]furan-2,8-diyl)bis(4-phenyl-1H-imidazole-5,2-diyl))dibenzoic acid (CMPD-A), dimethyl 4,4′-(5,5′-(dibenzo[b,d]furan-2,8-diyl)bis(4-phenyl-1H-imidazole-5,2-diyl))dibenzoate (CMPD-B), 4-(5-(dibenzo[b,d]furan-2-yl)-4-phenyl-1H-imidazol-2-yl)benzoic acid (CMPD-C), 4-amino-N⁵-[(2-chlorophenyl)methyl]-N³-cyclohexyl-N⁵-[2-(cyclohexylamino)-1-(5-methylfuran-2-yl)-2-oxoethyl]-1,2-thiazole-3,5-dicarboxamide (CMPD-D), 4-(4,5-diphenyl-1H-imidazol-2-yl)benzoic acid (CMPD-E), 4-amino-N5-benzyl-N5-(2-(benzylamino)-1-(5-methylfuran-2-yl)-2-oxoethyl)isothiazole-3,5-dicarboxamide (CMPD-G), 4-amino-N5-benzyl-N5-(2-((4-fluorobenzyl)amino)-1-(5-methylfuran-2-yl)-2-oxoethyl) isothiazole-3,5-dicarboxamide (CMPD-H), 4-amino-N5-(2-chlorobenzyl)-N5-(2-(cyclopentylamino)-1-(furan-2-yl)-2-oxoethyl)isothiazole-3,5-dicarboxamide (CMPD-J), 4-amino-N5-(2-chlorobenzyl)-N5-(2-(cyclohexylamino)-1-(5-methyl-furan-2-yl)-2-oxoethyl)isothiazole-3,5-dicarboxamide (CMPD-K), a mixture thereof, and a salt thereof

In one embodiment, the composition further comprises one or more anti-HIV drugs. In another embodiment, the one or more anti-HIV drugs are selected from the group consisting of HIV combination drugs, entry and fusion inhibitors, integrase inhibitors, non-nucleoside reverse transcriptase inhibitors, nucleoside reverse transcriptase inhibitors, and protease inhibitors.

In one embodiment, the subject is a mammal. In another embodiment, the subject is human.

The invention also includes a method of inhibiting, suppressing or preventing a viral infection in a subject in need thereof. The method comprises administering to the subject a composition comprising a therapeutically effective amount of at least one compound of Formula (I):

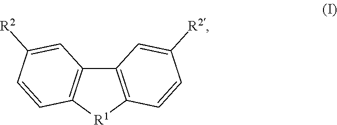

wherein in Formula (I):

R¹ is O, S, NH, N-alkyl, N—C(O)NH₂, N—CH₂C(O)NH₂, N—CH₂CH₂C(O)NH₂, CH₂, CH-alkyl, CH-OMe, CH-OEt, CH—C(O)NH₂, CH—CH₂C(O)NH₂, or CH—CH₂CH₂C(O)NH₂;

R² and R^{2′} are independently H or

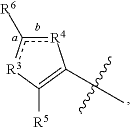

wherein,
(i) if ‘a’ is a double bond and ‘b’ is a single bond, then R³ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or
(ii) if ‘a’ is a single bond and ‘b’ is a double bond, then R³ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂; with the proviso that if R² is H then R^{2′} is not H; and

R⁵ and R⁶ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl, or a salt thereof,
wherein the viral infection comprises dengue fever, dengue hemorrhagic fever, dengue shock syndrome, West Nile virus infection, or respiratory syncytial virus infection.

In one embodiment, the compound of Formula (I) is a compound of Formula (Ia):

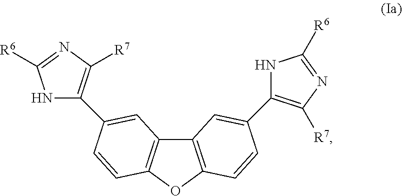

wherein in Formula (Ia):

R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

In one embodiment, the compound is selected from the group consisting of 4,4′-(5,5′-(dibenzo[b,d]furan-2,8-diyl)bis(4-phenyl-1H-imidazole-5,2-diyl))dibenzoic acid (CMPD-A), dimethyl 4,4′-(5,5′-(dibenzo[b,d]furan-2,8-diyl)bis(4-phenyl-1H-imidazole-5,2-diyl))dibenzoate (CMPD-B), a mixture thereof, and a salt thereof.

In one embodiment, the subject is a mammal In another embodiment, the subject is human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising panels A-D, is a series of schematic representations of the HIV-1 CA protein hexamer. Panel A is a schematic representation of the side view of a cross-linked hexamer. The NTC and CTD layers are indicated. Panel B is a schematic representation of the top-view of a cross-linked hexamer, with the positions of the first three helices of each protomer indicated by numbered circles. These form a helical barrel at the core of the hexamer. Panel C is a schematic representation of the top view of one sheet in the CcmK4-templated CA protein crystals, which recapitulates the hexameric lattice of authentic capsids at its planar limit. This view emphasizes that interactions between neighboring hexamers are mediated only by the CTD. Panel D is a schematic representation of the top view of the CTD-CTD interface that connects neighboring hexamers, as seen in the CcmK4-templated and cross-linked hexagonal crystals, and superimposed with the isolated full-affinity CTD dimer (Worthylake et al., 1999, Acta Crystallogr. D Biol. Crystallogr. 55(Pt 1):85-92). The black oval represents the twofold symmetry axis.

FIG. 2, comprising panels A-C, is a series of schematic representations of the NTD-NTD hexamerization interface. Panel A is a ribbon diagram of two adjacent HIV-1 CA proteins within hexameric arrangement illustrating the positions of the helices. Panel B is a schematic close-up of the residues that form the “hydrophobic core” between helices 1, 2, and 3. Panel C is a schematic close-up of residues in the NTD-NTD that comprise the interface. Residues that when mutated alter capsid assembly are highlighted.

FIG. 3 is a graph illustrating the inhibition of HIV-1 infection by compounds CK422 (CMPD-D) and CK026 (CMPD-A). FIG. 3A illustrates the effect of compounds on production of infectious single-round competent HIV-1NL4-3 virus. FIG. 3B illustrates the effect of compounds on the infection of recombinant luciferase-containing HIV-1 viruses (HIV-1NL4-3 backbone) pseudotyped with the envelope protein from HIV-1HxBc2. Virus infection is expressed as the percentage of infection (measured by luciferase activity in the target cells) observed in the presence of compound relative to the level of infection observed in the absence of the compound. The average data from three replicates are shown.

FIG. 4 is an image illustrating the sodium dodecyl sulfate-polyacrylamide gel electrophoresis (SDS-PAGE) analysis of wild-type (wt) and mutant HIV-1 CA proteins.

FIG. 5 is a graph illustrating the in vitro assembly kinetics of HIV-1 CA protein upon dilution into high-ionic-strength buffer.

FIG. 6 is a fluxogram illustrating development steps that may be used for development of HIV-1 CA protein hexamerization inhibitors or agonists.

FIG. 7, comprising FIGS. 7A-7C, is a series of graphs illustrating the effect of compound CMPD-A on the replication of single- and multiple-round infectious HIV-1. FIG. 7A is a graph illustrating disruption of infection by CMPD-A at early and post-entry stages as shown by single round infection assays. Illustrated are the effects of compound CMPD-A, on the infection of Cf2Th-CCR5 cells by recombinant luciferase-expressing HIV-1 bearing the envelope glycoprotein of the HIV-1_{YU-2} strain or amphotropic murine leukemia virus (AMLV). Virus infection was expressed as the percentage of infection (measured by luciferase activity in the target cells) observed in the presence of compound relative to the level of infection observed in the absence of the compound. The data from 3 replicates are shown. IC₅₀ value for compound CMPD-A against HIV-1 was demonstrated to be 33.3±0.31 μM. Compound CMPD-D was included as a compound control, as it has previously been determined not to have any effect on HIV-1 infection. FIG. 7B illustrates the effect of CMPD-A on replication of HIV-1_{IIIB} in primary peripheral HeLa P4-R5 MAGI cell line. FIG. 7C illustrates the finding that CMPD-A did not affect replication of HIV-1_{92BR030} in primary peripheral blood mononuclear cells (PBMC).

FIG. 8, comprising FIGS. 8A-8B, illustrates the proposed binding mode of CMPD-A to the HIV-1_{NL4-3} capsid protein. FIG. 8A is a surface representation of the monomeric unit of CA protein. CMPD-A, CMPD-E and a known CA inhibitor CAP-1 were docked to their predicted binding sites. FIG. 8B illustrates a schematic representation of proposed binding mode of CMPD-A in CA. Hydrogen bonded interactions are shown by arrows. The figure was generated using MOE ligX module.

FIG. 9 is a graph illustrating the comparison of the effects of compounds CMPD-A, CMPD-B, CMPD-C and CMPD-E on viral replication. Illustrated are the effects of compounds CMPD-A, CMPD-B, CMPD-C and CMPD-E on the infection of Cf2Th-CCR5 cells by recombinant luciferase-expressing HIV-1 bearing the envelope glycoprotein of the HIV-1_{YU-2} strain. Virus infection was expressed as the percentage of infection (measured by luciferase activity in the target cells) observed in the presence of compound relative to the level of infection observed in the absence of the compound. The data from 3 replicates are shown. IC₅₀ value for compound CMPD-E against HIV-1 was demonstrated to be 22.5±1.1 μM.

FIG. 10, comprising FIGS. 10A-10B, is a series of sensorgrams depicting the interaction of the (FIG. 10A) CMPD-E and (FIG. 10B) CMPD-F with sensor-chip immobilized HIV-1_{NL4-3} CA. CMPD-E at concentrations in the range 0.86-110 μM are shown. The individual rate constants were out of the dynamic range of the instrument. The equilibrium dissociation constants were as follows K_{D1}=66.3±4.8 μM; K_{D2}=66.3±5.2 μM. The chemical structures of each compound are shown inset.

FIG. 11, comprising FIGS. 11A-11B, illustrates experiments relating to binding of CMPD-E to CA. FIG. 11A illustrates the calorimetric titration of HIV-1_{NL4-3} CA with CMPD-E at 25° C. in Tris-HCl, 150 mM NaCl with 3% DMSO. The concentration of CA was 35 μM, and the syringe contained CMPD-E at a concentration of 600 μM. The experimental data fit with a binding model where two molecules of CMPD-E bind to one CA, each with a binding affinity of 25° C. is 85 μM, which corresponds to a change in Gibbs energy of −6.6 kcal/mol. The changes in enthalpy (ΔH) and entropy (ΔS) are −7.3 kcal/mol and −5.0 cal/(K×mol), respectively. FIG. 11B illustrates the temperature dependence of the enthalpy of binding of CMPD-E to CA. The slope corresponds to a heat capacity change of −220 cal/(K×mol).

FIG. 12, comprising FIGS. 12A-12B, illustrates representations of binding of CMPD-E with CA. FIG. 12A illustrates a comparison of the proposed binding site of CMPD-E with the binding site of compound PF74. Structural superpositioning of co-crystallized PF74 with NTD of CA protein on CA dimers. The protein is represented in cartoon model. The binding sites for PF74 and CMPD-E are distinct and opposite to each other. The van der Waals surface model of CMPD-E clearly shows CMPD-E sterically clashes with one of the CA protomers and hence blocks the assembly of the CA protein. FIG. 12 B illustrates the schematic representation of CMPD-E in the binding site of CA monomer. Hydrogen bonded interactions are shown by arrows. The figure was generated using MOE ligX module.

FIG. 13 is a graph illustrating the effect of CMPD-E on assembly of HIV-1 CA in vitro. CA assembly was monitored by an increase in turbidity using a spectrophotometer at 350 nm. CA was used at a final concentration of 30 μM, and CMPD-E at a final concentration of 147 μM. The presence of CMPD-E prevents the assembly of the capsid.

FIG. 14 is a bar graph illustrating the effect of mutation of capsid residues in and round the proposed CMPD-E binding site on compound binding. The interaction of CMPD-E at a concentration of 27.5 μM with wild-type and mutant versions of the CA protein was assessed using SPR. To allow comparison responses at equilibrium were normalized to the theoretical Rₘₐₓ, assuming a 2:1 interaction.

FIG. 15 is a graph illustrating the effect of CMPD-A on viral replication of DENV serotypes 1-4, yellow fever virus, and Japanese encephalitis virus. Illustrated are the effects of CMPD-A on the replication of DENY serotypes 1-4, yellow fever, and Japanese encephalitis virus in Vero E6 cells. The data from 3 replicates are shown. IC₅₀ values for CMPD-A against these flaviviruses are as follows: DENV1 IC₅₀=8.35 μM; DENV2 IC₅₀=1.43 μM; DENV3 IC₅₀=7.66 μM; DENV4 IC₅₀=2.37 μM.

FIG. 16 is a graph illustrating the effect of CMPD-A on viral replication of WNV. The effect of CMPD-A on the replication of WNV virus in Vero E6 cells is illustrated. The data from 2 replicates are shown. WNV IC₅₀=30 (±13) μM.

FIG. 17 is a graph illustrating the effect of CMPD-A on viral replication of respiratory syncytial virus (RSV). Illustrated are the effects of CMPD-A on the replication of RSV in Vero E6 cells. The data from two replicates are shown. The IC₅₀ for inhibition of RSV was determined to be 10.23 μM.

FIG. 18 is a fluxogram illustrating the Hybrid Structure-Based flow chart.

FIG. 19 is a graph illustrating the finding that CMPD-E displays broad antiviral activity against multiple subtypes of HIV-1.

FIG. 20, comprising FIGS. 20A-20C, illustrates the effect of analogues of CMPD-D (structures displayed in FIG. 20C) on HIV-1 virus production (FIG. 20A) and infection (FIG. 20B).

### DETAILED DESCRIPTION OF THE INVENTION

The present invention relates to the discovery that certain compounds are useful to treat or prevent HIV-1 viral infection in a vertebrate cell. These compounds bind to HIV-1 CA protein and act as antagonists or agonists of HIV-1 capsid hexamerization. These compounds inhibit or disturb one or more of the biological functions of the HIV-1 CA protein and therefore compromise the virus life cycle.

In one aspect, the invention provides a method of treating or preventing HIV-1 viral infection in a subject. The method comprises the step of administering the subject with a therapeutically effective amount of a pharmaceutical composition comprising a compound that disrupts one or more of the biological functions of the HIV-1 CA protein. In one embodiment, the subject is human.

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. “About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “CAP-1” refers to the compound [(N-(3-chloro-4-methylphenyl)-N′-[2-[([5-[(dimethylamino)-methyl]-2-furyl]-methyl)-sulfanyl]ethyl]urea] or a salt thereof.

As used herein, the term “CMPD-A” or “CK026” refers to the compound 4,4′-(4,4′-(dibenzo[b,d]furan-2,8-diyl)bis(5-phenyl-1H-imidazole-4,2-diyl))dibenzoic acid or a salt thereof.

As used herein, the term “CMPD-B” or “DMJ-I-073” refers to the compound dimethyl 4,4′-(4,4′-(dibenzo[b,d]furan-2,8-diyl)bis(5-phenyl-1H-imidazole-4,2-diyl))dibenzoate or a salt thereof.

As used herein, the term “CMPD-C” or “I-XW-091” refers to the compound 4-(5-(dibenzo[b,d]furan-2-yl)-4-phenyl-1H-imidazol-2-yl)benzoic acid or a salt thereof

As used herein, the term “CMPD-D” or “CK422” refers to the compound 4-amino-N5-(2-chlorobenzyl)-N3-cyclohexyl-N5-(2-(cyclohexylamino)-1-(5-methylfuran-2-yl)-2-oxoethyl)isothiazole-3,5-dicarboxamide or a salt thereof.

As used herein, the term “CMPD-E” or “I-XW-053” refers to the compound 4-(4,5-diphenyl-1H-imidazol-2-yl)benzoic acid or a salt thereof.

As used herein, the term “CMPD-F” or “NBD-556” refers to the compound N1-(4-chlorophenyl)-N2-(2,2,6,6-tetramethylpiperidin-4-yl)oxalamide or a salt thereof.

As used herein, the term “CMDP-G” or “CK292” refers to the compound 4-amino-N5-benzyl-N5-(2-(benzylamino)-1-(5-methylfuran-2-yl)-2-oxoethyl)isothiazole-3,5-dicarboxamide or a salt thereof.

As used herein, the term “CMPD-H” or “CK401” refers to the compound 4-amino-N5-benzyl-N5-(2-((4-fluorobenzyl)amino)-1-(5-methylfuran-2-yl)-2-oxoethyl)isothiazole-3,5-dicarboxamide or a salt thereof.

As used herein, the term “CMPD-J” or “CK551” refers to the compound 4-amino-N5-(2-chlorobenzyl)-N5-(2-(cyclopentylamino)-1-(furan-2-yl)-2-oxoethyl)isothiazole-3,5-dicarboxamide or a salt thereof.

As used herein, the term “CMPD-K” or “CK825” refers to the compound 4-amino-N5-(2-chlorobenzyl)-N5-(2-(cyclohexylamino)-1-(5-methylfuran-2-yl)-2-oxoethyl)isothiazole-3,5-dicarboxamide or a salt thereof.

As used herein, the term “polypeptide” refers to a polymer composed of amino acid residues, related naturally occurring structural variants, and synthetic non-naturally occurring analogs thereof linked via peptide bonds. Synthetic polypeptides may be synthesized, for example, using an automated polypeptide synthesizer. As used herein, the term “protein” typically refers to large polypeptides. As used herein, the term “peptide” typically refers to short polypeptides. Conventional notation is used herein to represent polypeptide sequences: the left-hand end of a polypeptide sequence is the amino-terminus, and the right-hand end of a polypeptide sequence is the carboxyl-terminus.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

As used herein, the term “antiviral agent” means a composition of matter which, when delivered to a cell, is capable of preventing replication of a virus in the cell, preventing infection of the cell by a virus, or reversing a physiological effect of infection of the cell by a virus. Antiviral agents are well known and described in the literature. By way of example, AZT (zidovudine, Retrovir®, Glaxosmithkline, Middlesex, UK) is an antiviral agent that is thought to prevent replication of HIV in human cells.

As used herein, the term “treatment” or “treating” is defined as the application or administration of a therapeutic agent, i.e., a compound useful within the invention (alone or in combination with another pharmaceutical agent), to a subject, or application or administration of a therapeutic agent to an isolated tissue or cell line from a subject (e.g., for diagnosis or ex vivo applications), who has an HIV-1 infection, a symptom of an HIV-1 infection or the potential to acquire an HIV-1 infection, with the purpose to cure, heal, alleviate, relieve, alter, remedy, ameliorate, improve or affect the HIV-1 infection, the symptoms of the HIV-1 infection or the potential to acquire the HIV-1 infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient” or “subject” refers to a human or a non-human animal. Non-human animals include, for example, livestock and pets, such as ovine, bovine, porcine, canine, feline and murine mammals. Preferably, the patient or subject is human.

As used herein, the terms “effective amount,” “pharmaceutically effective amount” and “therapeutically effective amount” refer to a non-toxic but sufficient amount of an agent to provide the desired biological result. That result can be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

As used herein, the term “instructional material” includes a publication, a recording, a diagram, or any other medium of expression that may be used to communicate the usefulness of the compounds useful within the invention. In some instances, the instructional material may be part of a kit useful for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the compounds useful within the invention or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound.

### Compositions

The composition of the invention comprises compounds that may be synthesized using techniques well-known in the art of organic synthesis.

In one embodiment, the composition of the invention comprises a compound selected from the group consisting of CMPD-A, CMPD-B, CMPD-C, CMPD-D, CMPD-E, CMPD-G, CMPD-H, CMPD-J, CMPD-K, a mixture thereof and a salt thereof

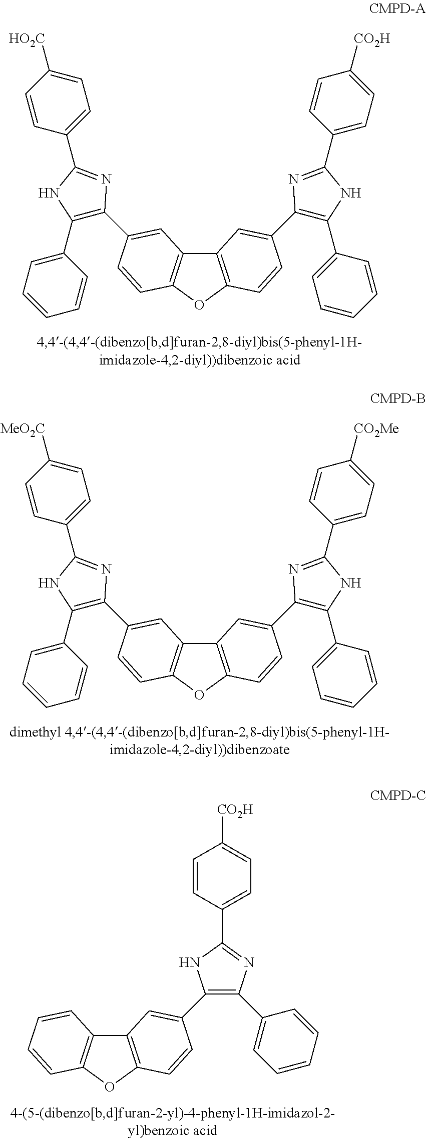 [SMILES: O=C(O)c1ccc(-c2nc(-c3ccc4oc5ccc(-c6nc(-c7ccc(C(=O)O)cc7)[nH]c6-c6ccccc6)cc5c4c3)c(-c3ccccc3)[nH]2)cc1 ; COC(=O)c1ccc(-c2nc(-c3ccc4oc5ccc(-c6nc(-c7ccc(C(=O)OC)cc7)[nH]c6-c6ccccc6)cc5c4c3)c(-c3ccccc3)[nH]2)cc1 ; O=C(O)c1ccc(-c2nc(-c3ccccc3)c(-c3ccc4oc5ccccc5c4c3)[nH]2)cc1]

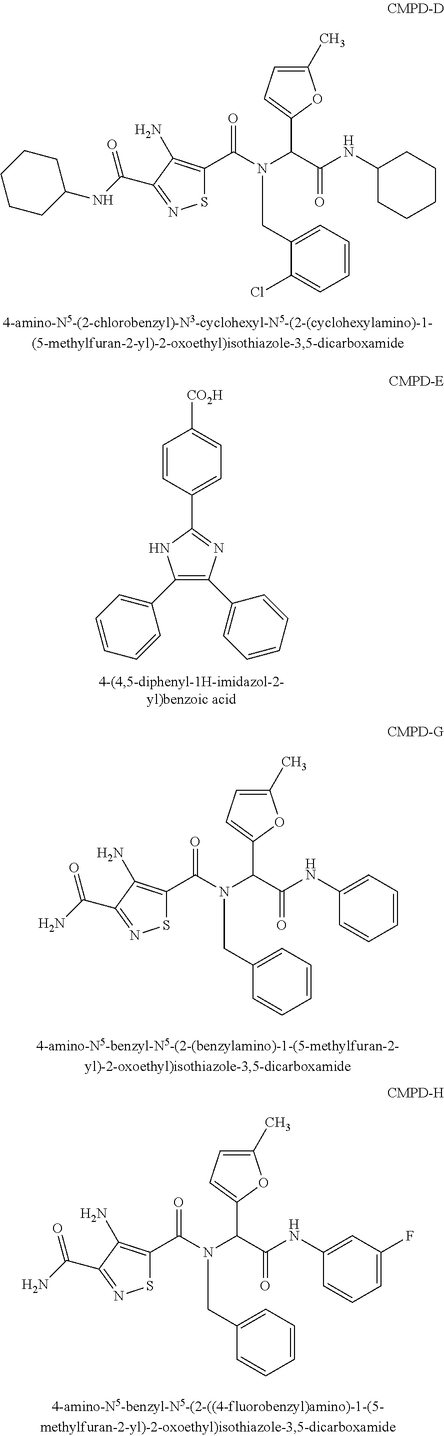 [SMILES: Cc1ccc(C(C(=O)NC2CCCCC2)N(Cc2ccccc2Cl)C(=O)c2snc(C(=O)NC3CCCCC3)c2N)o1 ; O=C(O)c1ccc(-c2nc(-c3ccccc3)c(-c3ccccc3)[nH]2)cc1 ; Cc1ccc(C(C(=O)Nc2ccccc2)N(Cc2ccccc2)C(=O)c2snc(C(N)=O)c2N)o1 ; Cc1ccc(C(C(=O)Nc2cccc(F)c2)N(Cc2ccccc2)C(=O)c2snc(C(N)=O)c2N)o1]

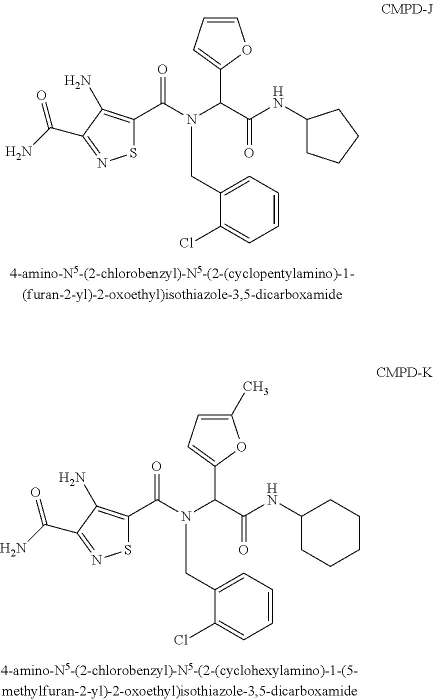 [SMILES: NC(=O)c1nsc(C(=O)N(Cc2ccccc2Cl)C(C(=O)NC2CCCC2)c2ccco2)c1N ; Cc1ccc(C(C(=O)NC2CCCCC2)N(Cc2ccccc2Cl)C(=O)c2snc(C(N)=O)c2N)o1]

In one embodiment, the composition of the invention comprises a compound of Formula (I):

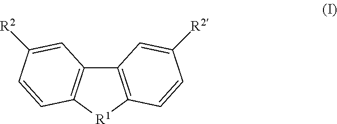

wherein in Formula (I),

R¹ is O, S, NH, N-alkyl, N—C(O)NH₂, N—CH₂C(O)NH₂, N—CH₂CH₂C(O)NH₂, CH₂, CH-alkyl, CH-OMe, CH-OEt, CH—C(O)NH₂, CH—CH₂C(O)NH₂, or CH—CH₂CH₂C(O)NH₂;

R² and R^{2′} are independently H or

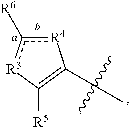

wherein,

(i) if ‘a’ is a double bond and ‘b’ is a single bond, then R³ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or

(ii) if ‘a’ is a single bond and ‘b’ is a double bond, then R³ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂; with the proviso that if R² is H then R^{2′} are not H; and

R⁵ and R⁶ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

In another embodiment, the compound of Formula (I) is a compound of Formula (Ia):

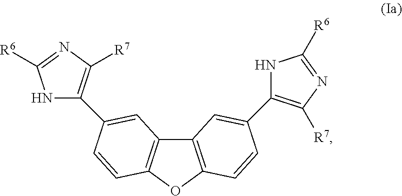

wherein in Formula (Ia),

R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

In yet another embodiment, in Formula (Ia) R⁶ and R⁷ are independently aryl or substituted aryl, or a pharmaceutically acceptable salt thereof

In yet another embodiment, the compound is selected from the group consisting of 4,4′-(5,5′-(dibenzo[b,d]furan-2,8-diyl)bis(4-phenyl-1H-imidazole-5,2-diyl))dibenzoic acid (CMPD-A), dimethyl 4,4′-(5,5′-(dibenzo[b,d]furan-2,8-diyl)bis(4-phenyl-1H-imidazole-5,2-diyl))dibenzoate (CMPD-B), a mixture thereof and a salt thereof.

In yet another embodiment, the compound of Formula (I) is a compound of Formula (Ib):

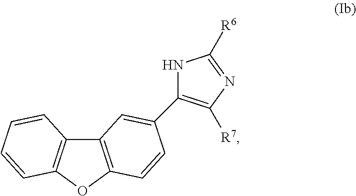

wherein in Formula (Ib) R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

In yet another embodiment, in Formula (Ib), R⁶ and R⁷ are independently aryl or substituted aryl, or a pharmaceutically acceptable salt thereof.

In yet another embodiment, the compound is 4-(5-(dibenzo[b,d]furan-2-yl)-4-phenyl-1H-imidazol-2-yl)benzoic acid (CMPD-C), or a pharmaceutically acceptable salt thereof.

In one embodiment, the composition of the invention comprises a compound of Formula (II),

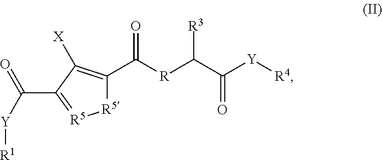

wherein:

R is NR₂, CHR₂, O or S;

R¹, R², R³ and R⁴ are independently H, alkyl, substituted alkyl, cycloalkyl, substituted cycloalkyl, aryl, substituted aryl, benzyl, substituted benzyl, heteroaryl, or substituted heteroaryl;

R⁵ is N or CH;

R^{5′} is CH₂, NH, S or O;

X is —NH₂, —NHR¹, —NR¹R², —OH, cyano, alkyl, alkoxy, halogen, sulfonamide, aryl, substituted aryl, heteroaryl or substituted heteroaryl; and,

each occurrence of Y is independently NH, NR¹, O, CH₂, CHR¹ or CR¹R²;

or a pharmaceutically acceptable salt thereof.

In another embodiment, the composition of the invention comprises a compound of Formula (II),

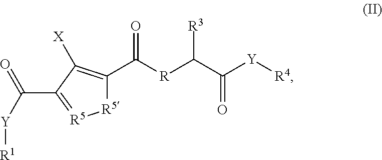

wherein:

R is NR₂, CHR₂, O or S;

R¹, R², R³ and R⁴ are independently H, alkyl, substituted alkyl, cycloalkyl, substituted cycloalkyl, aryl, substituted aryl, benzyl, substituted benzyl, heteroaryl, or substituted heteroaryl;

R⁵ is N or CH;

R^{5′} is CH₂, NH, S or O;

X is —NH₂, —NHR¹, —NR¹R², —OH, cyano, alkyl, alkoxy, or halogen; and,

each occurrence of Y is independently NH, NR¹, O, CH₂, CHR¹ or CR¹R²;

or a pharmaceutically acceptable salt thereof.

In yet another embodiment, the composition of the invention comprises a compound of Formula (II),

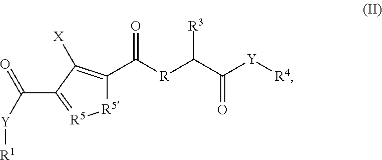

wherein:

R is NR₂, or CHR₂;

R¹, R², R³ and R⁴ are independently H, alkyl, substituted alkyl, cycloalkyl, substituted cycloalkyl, aryl, substituted aryl, benzyl, substituted benzyl, heteroaryl, or substituted heteroaryl;

R⁵ is N or CH;

R^{5′} is CH₂, NH, S or O;

X is —NH₂, —NHR¹, —NR¹R², —OH, cyano, alkyl, alkoxy, or halogen; and,

each occurrence of Y is independently NH, NR¹, O, CH₂, CHR¹ or CR¹R²;

or a pharmaceutically acceptable salt thereof.

In yet another embodiment, the compound is selected from the group consisting of 4-amino-N⁵-[(2-chlorophenyl)methyl]-N³-cyclohexyl-N⁵-[2-(cyclohexylamino)-1-(5-methylfuran-2-yl)-2-oxoethyl]-1,2-thiazole-3,5-dicarboxamide (CMPD-D), 4-amino-N5-benzyl-N5-(2-(benzylamino)-1-(5-methylfuran-2-yl)-2-oxoethyl)isothiazole-3,5-dicarboxamide (CMPD-G), 4-amino-N5-benzyl-N5-(2-((4-fluorobenzyl)amino)-1-(5-methylfuran-2-yl)-2-oxoethyl) isothiazole-3,5-dicarboxamide (CMPD-H), 4-amino-N5-(2-chlorobenzyl)-N5-(2-(cyclopentylamino)-1-(furan-2-yl)-2-oxoethyl)isothiazole-3,5-dicarboxamide (CMPD-J), 4-amino-N5-(2-chlorobenzyl)-N5-(2-(cyclohexylamino)-1-(5-methyl-furan-2-yl)-2-oxoethyl)isothiazole-3,5-dicarboxamide (CMPD-K), a mixture thereof, or a pharmaceutically acceptable salt thereof.

In one embodiment, the compound useful in the invention is a compound of Formula (III),

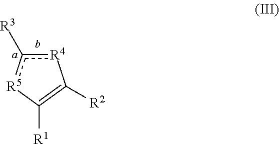

wherein:

R¹, R² and R³ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl, and

R⁴ and R⁵ are such that:

(i) if ‘a’ is a double bond and ‘b’ is a single bond, then R⁵ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or

(ii) if ‘a’ is a single bond and ‘b’ is a double bond, then R⁵ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂;

or a pharmaceutically acceptable salt thereof.

In another embodiment, in Formula (III) R⁴ and R⁵ are such that:

(i) if ‘a’ is a double bond and ‘b’ is a single bond, then R⁵ is N, CH, C—OMe, C—OEt, C—C(O)NH₂, C—CH₂C(O)NH₂ or C—CH₂CH₂C(O)NH₂, and R⁴ is NH, N-alkyl, N—C(O)NH₂, N—CH₂C(O)NH₂ or N—CH₂CH₂C(O)NH₂, or

(ii) if ‘a’ is a single bond and ‘b’ is a double bond, then R⁵ is NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂.

In yet another embodiment, in Formula (III) R⁴ and R⁵ are such that:

(i) if ‘a’ is a double bond and ‘b’ is a single bond, then R⁵ is N or CH, and R⁴ is NH or N-alkyl, or

(ii) if ‘a’ is a single bond and ‘b’ is a double bond, then R⁵ is NH or N-alkyl, and R⁴ is N or CH.

In yet another embodiment, in Formula (III) R⁴ and R⁵ are such that:

(i) if ‘a’ is a double bond and ‘b’ is a single bond, then R⁵ is N, and R⁴ is NH or N-alkyl, or

(ii) if ‘a’ is a single bond and ‘b’ is a double bond, then R⁵ is NH or N-alkyl, and R⁴ is N.

In yet another embodiment, the compound is 4-(4,5-diphenyl-1H-imidazol-2-yl)benzoic acid (CMPD-E) or a salt thereof.

The term “alkyl” refers to saturated aliphatic groups, including straight-chain alkyl groups, branched-chain alkyl groups, cycloalkyl, heterocyclyl, cycloalkyl (alicyclic) groups, alkyl substituted cycloalkyl groups, and cycloalkyl substituted alkyl groups. In preferred embodiments, a straight chain or branched chain alkyl has 6 or fewer carbon atoms in its backbone (e.g., C₁-C₆ for straight chain, C₃-C₆ for branched chain), and more preferably has 6 or fewer carbon atoms in the backbone. Likewise, preferred cycloalkyls have from 3-6 carbon atoms in their ring structure. Moreover, alkyl (such as methyl, ethyl, propyl, butyl, pentyl, and hexyl) include both “unsubstituted alkyl” and “substituted alkyl”, the latter of which refers to alkyl moieties having substituents replacing a hydrogen on one or more carbons of the hydrocarbon backbone, which allow the molecule to perform its intended function.

The term “substituted aryl” or “substituted heteroaryl” is aryl or heteroaryl substituted with one or more substituents independently selected from the group consisting of halogen, (C₁-C₆)alkyl, —(C₁-C₃)alkylene-R⁸, —OR⁸, —O(C₁-C₃)alkylene-R⁸, (C₁-C₃)fluoroalkoxy, —NO₂, —C≡N, —C(═O)—(C₁-C₃)alkyl, —C(═O)OR⁸, —C(═O)NR⁸₂, —C(═NR⁹)NR⁸₂, —(C₁-C₃)alkylene-C(═O)OR⁸, —O(C₁-C₃)alkylene-C(═O)OR⁸, —(C₁-C₆)alkylene-OR⁸, —NR⁸₂, —P(═O)(OR⁸)₂, —OP(═O)(OR⁸)₂, —S(C₁-C₆)alkyl, —S(O)(C₁-C₆)alkyl, —SO₂(C₁-C₆)alkyl, —SO₂NR⁸₂, —OSO₂(C₁-C₆)alkyl, —OSO₂R⁸, —NHC(═O)(C₁-C₆)alkyl, —OC(═O)(C₁-C₃)alkyl, —O(C₂-C₆)alkylene-NR⁸₂ and (C₁-C₃)perfluoroalkyl, wherein R⁸ and R⁹ in each occurrence are independently H, C₁-C₃ alkyl, substituted C₁-C₃ alkyl, aryl, or substituted aryl. In one embodiment, the substituted aryl or heteroaryl has at least one substituent selected from the group consisting of halogen, —OR⁸, —NO₂, —C≡N, —C(═O)OR⁸, and —C(═O)NR⁸₂, wherein each occurrence of R⁸ is independently H, C₁₋₃ alkyl, substituted C₁₋₃ alkyl, aryl, or substituted aryl. In another embodiment, the substituted aryl or heteroaryl has at least one COOH substituent.

### Identification of HIV-1 CA Protein Hexamerization Antagonists or Agonists

The NTD of the HIV-1 CA protein plays a role in forming the hexameric lattice formation that is required for correct assembly of the HIV-1 CA protein. In addition to playing a role in hexameric lattice assembly, the stability of the NTD-NTD interface regulates the correct temporal series of replicative events after fusion, such as reverse transcription. Accordingly, mutational studies have demonstrated that mutations that stabilize or destabilize interactions within the capsid shell or reduce the rate at which the CA proteins polymerize are detrimental to the virus. Capsid shells that are unstable do not form infectious virions, and those that are either slightly too unstable or stable compared to the wild-type CA protein do not enter into reverse transcription correctly and hence cannot effectively integrate the HIV-1 provirus.

A compound that recapitulates the effects of mutation, by inhibiting assembly, accelerating disassembly, or artificially stabilizing the capsid shell, should attenuate or even kill the virus. In one aspect, such a compound binds at the NTD-NTD interface. These hexamerization antagonists or agonists represent a new class of small-molecule HIV-1 CA protein inhibitors, targeted at a highly conserved oligomerization surface and possessing a novel mechanism of action.

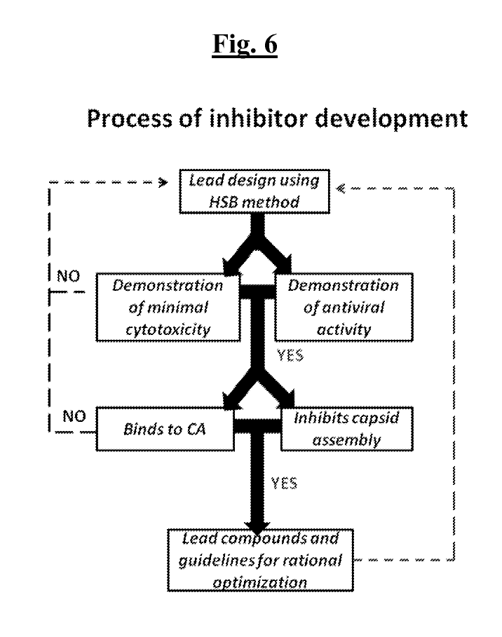

Novel small molecules that disrupt the HIV-1 CA protein hexamerization interface may be identified using a cross-disciplinary approach, combining novel computational methods of compound identification with newly developed biochemical and virology assays. The data obtained may then be used to direct the iterative redesign and chemical synthesis of novel lead compounds. The steps in inhibitor development that may be used for development of an inhibitor or agonist of HIV-1 CA protein hexamerization are illustrated schematically in FIG. 6.

### Computational Screening and Design of Small-Molecule Inhibitors Against the NTD-NTD Hexamerization Interface Using the Hybrid Structure-Based (HSB) Method

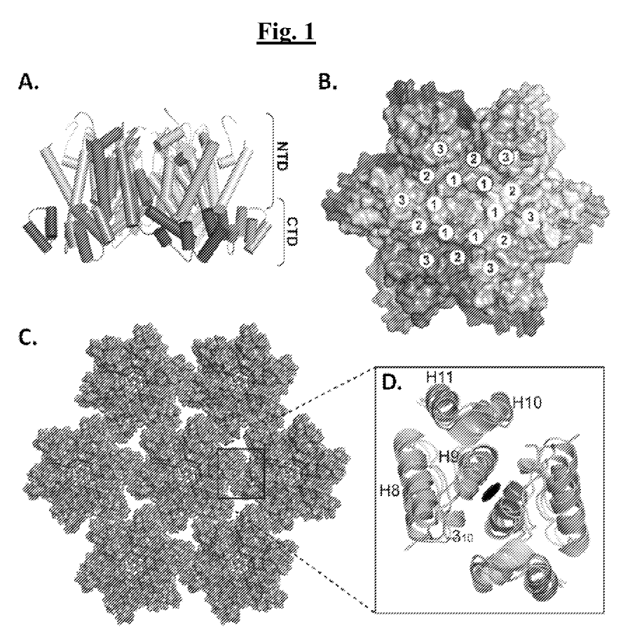

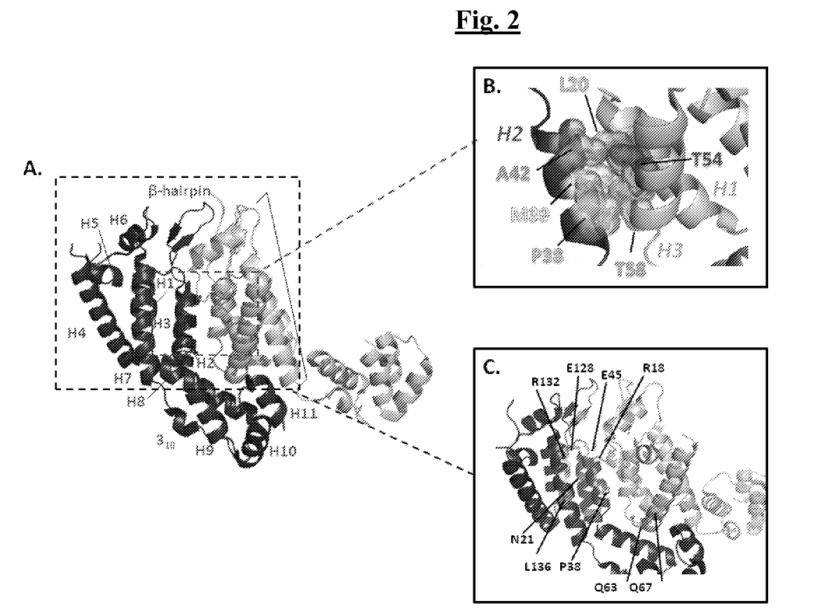

The capsid of the HIV-1 virus has a distinct geometry of a fullerene cone consisting of nearly 250 hexamers and 12 pentamers of the viral CA protein (FIG. 1). The hexagonal capsid lattice is composed of three different interfaces: an NTD-NTD interface that has six-fold symmetry and forms the hexameric ring; an NTD-CTD interface between adjacent monomers; and a homodimeric CTD-CTD interface. The ring formed by the interactions of adjacent NTDs displays a higher level of rigidity than the outer ring of CTDs. The interface between two adjacent CA protein monomers within the hexameric configuration (FIG. 2) displays qualities consistent with the relatively weak affinity: it has a small interface area (˜1,140 Å²) and low complementarity. The hexamer interface is primarily formed by polar interactions, with only a small number of hydrophobic contacts. The interface is highly hydrated, with the water molecules contributing to the formation of a pervasive hydrogen-bonding network between HIV-1 CA proteins. Mutagenesis studies within the NTD of CA protein have shown that the hexamer interface is very sensitive to genetic perturbation—single point mutations can lead to a number of altered CA proteins, each of which is damaging to the virus that harbors them. These combined characteristics of the NTD-NTD interface lend it perfectly to targeting by small-molecule inhibitors. Targeting protein-protein interactions for a therapeutic purpose is an attractive idea that has proved to be extremely challenging in practice using standard methods of computational screening. The large and flat landscape of most contact surfaces makes them less amenable to intervention by a small molecule. In recent years, however, growing evidence has demonstrated that small molecules can disrupt such large and complex protein interactions by binding to interface “hot spots” with drug-like potencies (Wells & McClendon, 2007, Nature 450(7172):1001-1009). The HSB method utilizes the information in such “hot spots” to inform the screening procedure. The HSB method may thus be used to design small-molecule inhibitors targeted to the NTD-NTD hexameric interface of the HIV-1 CA protein.

The HSB method combines the best elements of two virtual screening strategies: (1) ligand-based methods and (2) structure-based methods. The method uses ligand-based methods to build enriched libraries of small molecules, and then employs a combined receptor-ligand pharmacophore to screen molecules from the enriched library and to further dock the molecules to their receptor. The docked complexes are then scored based on a number of physico-chemical parameters to indicate high-ranking molecules. The results of this detailed analysis of the dynamic mode of association between the receptor and ligand are then used to list candidate molecules that are suitable for biological and biochemical testing. The HSB method is iterative, and information derived from biological and biochemical studies is used to improve lead design and optimize favorable characteristics (Kortagere & Welsh, 2006, J. Comput. Aided Mol. Des. 20(12):789-802). A description of the application of the HSB method to designing inhibitors of NTD-NTD interface follows.

### Screening of a Novel Enriched Database of Small Molecules Using the HSB Method and the Structure of the HIV-1 CA Protein:

The first phase in the HSB method is the development of a comprehensive electronic database of commercially available small molecules. The next phase of the HSB method is the generation of the combined ligand-protein pharmacophore (also called the hybrid pharmacophore). In this phase, the pharmacophore is customized to capture the essential features of interactions occurring at the hexamerization interface of the CA_{NTD}. A model of the CA protein complex is prepared from a x-ray-structure or NMR-derived structure and energy minimized as appropriate. The combined pharmacophore is then designed centered around those residues responsible for the stability of the interface. The database is then screened against the pharmacophore and first filtered according to Lipinski's “rule of five” to identify “drug-like” molecules or to the blood-brain barrier (BBB) penetration model. The full set of docked structures may then be energy minimized using a standard molecular modeling package, such as SYBYL. The best ranking complexes may then be visually inspected to include compounds that maximize the inhibition of the NTD-NTD interface.

The docking program proposed above provides some level of receptor flexibility at the binding site. However, a complete induced-fit model cannot be achieved using this level of screening as it is computationally expensive. This aspect may be addressed by using a docking program called Glide (Schrödinger, New York, N.Y.) that has been demonstrated to be useful in the evaluation of the final best docked molecules. This method ensures that the best docked complexes are appropriately redocked and rescored. Since no single docking or scoring program may efficiently capture the intricacies of the docking process, the process of using more than one docking program ensures that the best ranked molecules that are short listed for experimental validation are also screened efficiently. Lead molecules identified from the biochemical screening may be used as query molecules in the iterative HSB method to develop a complete structure-activity relationship.

### In Vitro Validation of the Antiviral Activity of the HSB-Identified Compounds

Several HIV-1 CA protein in vitro assembly assays may be used to test the anti-assembly properties of the compounds useful within the invention. The potential antiviral effects of the compounds identified from the HSB screen may be evaluated in both single- and multiple-round infection assays and using cells relevant to HIV-1 pathogenesis. Characterization of the compounds in both assembly and antiviral assays allows for the assessment of the effect of the compounds on the functional oligomeric HIV-1 CA/Gag. The cellular toxicity of the compounds, as well as the effects of mutation of the putative compound binding site within CA protein on their antiviral efficacy, may also be determined

### Cell Viability and Cytotoxicity Assays:

Compounds identified in the described assays are screened in target cells to identify compounds with undesirable levels of cytotoxicity, which may therefore be unsuitable as drug candidates. Compound cytotoxicity may also affect the results of the antiviral activity assays.

Compounds are assayed for cytotoxicity using concentrations (in half-log increments) low enough to be completely non-toxic and, if possible, high enough to result in complete cell death. Exposure times should include, in non-limiting examples, 10 minutes, 2 hours, 24 hours, and 8 days, and any and all whole or partial increments therebetween. The 8-day exposure may reveal levels of cytotoxicity that may affect the multiple-round infection assay described below. Compounds that demonstrate high levels of cytotoxicity should not be considered for further evaluation.

### In Vitro Antiviral Efficacy:

The potential antiviral activity of compounds identified in the HSB screen is assessed using a variety of cellular infection assays and HIV-1 isolates as will now be described.

### Single-Round Infection Assay:

A single-round infection assay may be used to determine whether the compounds affect early events (such as uncoating) or late events (such as assembly) or both. The single-round infection assay has been used for studies of inhibitors of HIV-1 replication (Si et al., 2004, Proc. Natl. Acad. Sci. U.S.A. 101(14):5036-5041). Effects on assembly are identified by incubating the viral producer cells in the presence of the compound. Virus particles are purified from the supernatants of the producer cells and used to infect the target cells. Aberrant assembly is then manifested as a decrease in infectivity within the target cells. Similarly, uncoating effects may be determined by producing virus in the absence of compound, then infecting target cells in the presence of compounds. Once the single-round infection assay identifies a compound with potent antiviral activity, additional virologic and biochemical experiments described herein may be performed using this compound to clarify its mechanism of action, to determine its specificity to HIV-1, and to address whether it may also affect viral assembly.

### Infection of Peripheral Blood Mononuclear Cells (PBMCs):

The antiviral activity of the test compounds identified from the single-round infection assay are verified using infectious HIV-1 derived from infectious molecular clones (IMCs) and assessing virus replication in peripheral blood mononuclear cells (PMBCs). Examples of virus that may be used in such research are molecularly cloned, infectious viruses derived from the NL4-3, YU-2, ADA, and BaL primary macrophage-tropic isolates, the 89.6 and ELI dual-tropic isolates, and the HXBc2 laboratory-adapted virus. In addition, viral stocks of three subtype A isolates (KNH1144 and KNH1207, both R5 utilizing; and 96USNG17, X4 utilizing), two subtype C isolates (93MW965 and SM145, both R5 utilizing), and one EA isolate (CM240, R5 utilizing) may also be produced by transfection of IMCs (NIH AIDS Reagent and Reference Program) and used to infect PBMCs. Supernatants of these cells are assessed for the amount of virus by reverse transcriptase assay. Equivalent amounts of virus are incubated with human PBMCs in the presence of increasing amounts of test compound. HIV-1 replication is then followed by periodic measurement of viral reverse transcriptase in culture supernatants. The effects of the compound on the replication of simian immunodeficiency virus and/or amphotropic murine leukemia virus are determined in parallel, allowing an assessment of the specificity of any observed effects.

Generation of HIV-1 Escape Variants from the Antiviral Effects of the Compounds Useful within the Invention:

The generation of HIV-1 variants in tissue culture systems that are resistant to the inhibitory effects of the compounds identified in the assays may provide insights into the compound binding/mechanism that complement the studies proposed above. The study of the development and molecular basis of resistance to test compounds may employ well-characterized primary HIV-1 isolates. IMCs are available for both the YU-2 and ADA isolates (Gendelman et al., 1988, J. Exp. Med. 167(4):1428-1441; Li et al., 1991, J. Virol. 65(8):3973-3985). The YU-2 provirus was directly cloned from the brain of an HIV-1-infected individual and therefore has never been subjected to the potential selection imposed by passage of the virus in tissue culture (Li et al., 1991, J. Virol. 65(8):3973-3985). The ADA virus was minimally passaged in peripheral blood monocytes prior to molecular cloning (Gendelman et al., 1988, J. Exp. Med. 167(4):1428-1441). Both YU-2 and ADA viruses are R5 (macrophage-tropic) and are representative of the clinically most abundant viruses.

The analysis of resistance may be undertaken for compounds that exhibit reasonable potency and breadth of activity. The study of resistance to such broadly active compounds is more likely to provide insight into general mechanisms for the emergence of resistance to small-molecule inhibitors of CA protein-CA protein interactions. Furthermore, only compounds with sufficient potency and breadth are likely to serve as components of clinically useful modalities.

The analysis of compound resistance is performed in parallel with two viruses, e.g., the YU-2 and ADA viruses. The use of two primary viruses allows assessment of the potential generality of results obtained. The YU-2 experiments are illustrated herein, with the understanding that the experiments with ADA may be performed in an identical manner.

The YU-2 infectious provirus is transfected by electroporation into human PBMCs and the resultant virus is propagated in these cells. PBMCs from a single donor are used throughout these experiments to avoid potential variables associated with the replication of virus in different target cells. Different concentrations of the compound are added to several parallel cultures, and virus replication is assessed by reverse transcriptase. Virus that is detectable in the culture using the highest compound concentration is propagated in two subsequent cultures, one with the same compound concentration and one with the next highest compound concentration. This process is repeated until any further increase in the compound concentration results in virus inhibition or cell toxicity. At this point, biological clones of the putative resistant virus are prepared by end-point dilution. The biological clones are tested for compound sensitivity, alongside a control YU-2 virus that has been passaged comparably in the absence of compound.

The biologically cloned compound-resistant YU-2 viruses (herein designated YU-2R) may be characterized at the molecular level, focusing on changes in the gag gene. If subsequent studies indicate that YU-2R components other than gag contribute to compound resistance, other proviral genomic regions may be studied using similar approaches. To identify mutations conferring resistance, chromosomal DNA is extracted from the infected cells and the HIV-1 gag sequence is amplified by polymerase chain reaction (PCR) for sequence analysis. Three PCR clones from each of two independent PCR amplifications are sequenced in their entirety, further allowing a distinction between clone- or PCR-specific changes and changes potentially responsible for compound resistance.

The relevance of predicted amino acid changes in the YU-2R Gag protein compound resistance in the context of viral replication are then determined Each of the amplified gag segments are introduced either singly or in combination into the original YU-2 IMC and the cumulative effects of these changes on compound resistance are assessed.

Sequence comparison of the YU-2R gag clones that allow relative resistance to compounds in this assay may identify predicted amino acid changes in the Gag polyprotein common to all resistance-associated clones. Examination of the location of altered amino acids in available structures of the individual domains of Gag (MA protein, CA protein, and NC) (Massiah et al., 1994, J. Mol. Biol. 244(2):198-223; Morellet et al., 1992, EMBO J. 11(8):3059-3065) may provide clues regarding the likely importance of some of the changes, given the understanding of the compound binding site and potential mechanism of compound inhibition derived from the in vitro antiviral activity studies and biochemical and structural characterization studies. Through site-directed mutagenesis, these consensus changes, individually or in combination, may be introduced into prokaryotic expression vectors that drive the overproduction of the YU-2 MA and CA proteins. The NC protein is small enough to be amenable to peptide synthesis (Morellet et al., 1992, EMBO J 11(8):3059-3065). A likely explanation for resistance is a decrease in the affinity of YU-2R CA protein for the compound, compared with the parental YU-2 CA protein. This assertion can be directly tested using the SPR-based binding assay outlined below. However, an alternative explanation for resistance to a compound is that the virus alters the rate of capsid assembly. This possibility may be investigated using the in vitro CA protein assembly assay, also outlined below (Li et al., 2009, J. Virol. 83(21):10951-62). If resistance to a compound entails a modification of the drug's binding site on the CA protein, this may be accompanied by a decrease in the replicative capacity of the virus, due to the conserved nature of the residues at the hexamerization interface. Such a decrease should be detectable in the aforementioned fully infectious and single-round replication assays.

### Data Analysis:

In one aspect, the studies described herein identify the specific capsid amino acid changes responsible for the development of resistance to the compounds useful within the invention. Insight into the mechanisms of such resistance may be thus obtained. An appreciation of the molecular pathways used by HIV-1 to achieve compound resistance will be useful in several ways: (1) Analysis of sequence changes found in naturally occurring HIV-1 may be performed with the purpose of identifying potentially rare variants that are spontaneously resistant to compounds; (2) If multiple compound molecules with potency and breadth sufficient for clinical utility become available, the rapidity with which HIV-1 develops resistance to individual compounds may be compared side-by-side—this comparison may help prioritize analogs for clinical development; in addition, studies of the compounds in combination may be useful in optimizing their potential in a clinical setting; (3) Ways of designing test compound analogs able to inhibit resistant viruses may become apparent, allowing second-generation compounds with greater breadth and/or efficacy to be developed.

### Biochemical Verification of the CA-Binding Properties, Anti-Assembly Activity of the HSB-Identified Compounds, and Structural Investigations of Inhibitor-CA Protein Complexes

The molecules identified in the HSB screen are predicted to bind to the NTD of HIV-1 CA and thereby alter its assembly of this protein. However, it is possible that the compounds may also interfere with HIV-1 infection through mechanisms not involving the CA protein.

Determining whether a compound targets the HIV-1 CA protein may be achieved by its CA protein direct binding and anti-assembly properties using SPR and in vitro assembly assays, respectively. These biochemical assays may also be used to determine how compounds interact with CA protein, and thus whether these compounds stabilize or destabilize the molecular assemble. Structural investigations of small molecule-CA protein complexes may also be performed. This structural analysis may reveal elements that can be exploited to improve binding affinity and offer further insights into their mechanism of action.

### Wild-Type and Mutant HIV-1 CA:

The P90A mutation reduces the affinity of CypA for CA protein and the A92E mutation arises upon selection of HIV-1 in HeLa cells treated with a cyclosporin A analogue. The double mutants P90A/A92 and P90E/A92E are herein designated AE and EE for simplicity. N-terminal extension of the CA protein causes a change in HIV-1 CA protein morphology from mature-like tubes to immature-like spheres (von Schwedler et al., 1998, EMBO J. 17(6):1555-1568), by abrogating the positive charge of proline 1 such that it cannot make a salt bridge with a buried aspartic acid side chain (Asp51) (Gitti et al., 1996, Science 273(5272):231-235). These facts may be used to determine whether the compounds useful within the invention may disrupt the assembly of immature capsids only, mature capsids only, or both. Additional mutants needed in the course of the study may be generated by site-directed mutagenesis. Capsid proteins from the non-subtype B strains used to gauge breadth of efficacy of the CA-directed compounds may also be cloned, overexpressed, and purified using recombinant techniques.

### In Vitro Assembly of HIV-1 CA:

Soluble HIV-1 CA protein can be triggered to assemble into tubes similar in diameter and morphology to intact cores by dilution into high-ionic-strength buffer. The kinetics of assembly can be followed by monitoring the increase in turbidity using a spectrophotometer.

Direct Binding of HSB-Identified Compounds using SPR:

Monitoring the interactions of small molecule compounds with their protein targets using SPR interaction analysis is becoming more commonplace in the iterative process of therapeutic design. Optical biosensors such as the Biacore series (GE Healthcare Life Sciences, Little Chalfont, Buckinghamshire, UK) are able to monitor the affinity (K_{A}, K_{D}) and kinetics (kₒₙ, k_{off}) of a particular interaction using a minimum of material. The direct binding of HSB-identified compounds to HIV-1 may thus be analyzed using these methods.

SPR interaction analyses of wild-type and mutant HIV-1 CA proteins are performed on a Biacore 3000 optical biosensor or a ProteON XPR36 array with simultaneous monitoring of four flow cells. Immobilization of HIV-1 CA protein to sensor chips may be achieved using standard amine coupling as per the manufacturer's protocols. A blank surface is used to correct for background binding and instrument and buffer artifacts. Direct binding experiments of small molecules to the HIV-1 CA protein is assessed by injecting increasing concentrations of the compounds over a surface containing the immobilized HIV-1 CA protein to determine affinity, kinetics, and stoichiometry. The density, flow rate, buffer, and regeneration conditions may be determined experimentally. Design and use of the SPR assay thus provide several parameters important for the further development of CA-targeted antiviral compounds, including specificity, affinity, stoichiometry, and more importantly association (kₒₙ) and dissociation (k_{off}) rate constants. In addition, this assay may also be used to interrogate the compound binding site on the HIV-1 CA protein.

SPR data analysis is performed using Biaevaluation 4.0 software (Biacore). The average kinetic parameters (association [kₐ] and dissociation [k_{d}] rates) generated from a minimum of four data sets will be used to define equilibrium dissociation constants (K_{D}).

There may be potential limitations with the SPR assays designed to monitor the direct binding of small molecules to HIV-1 CA protein. The solubility of the compounds in DMSO may be above the tolerance of the instrument. The Biacore 3000 biosensor has a DMSO tolerance of up to 8% and the ProteOn XPR36 has a tolerance of up to 10%. The predicted physical-chemical properties of these compounds suggest that they will be soluble in DMSO concentrations within the functional range of the instrument. If the molecules require more than 8% or 10% organic solvent to be soluble, alternative solubilizations may be employed. The sensitivity of the instrument should also be sufficient to detect binding of such small molecules to the target protein. Numerous studies indeed document the use of SPR to measure interaction of small-molecule ligand as small as 100 Da with protein targets (Bravman et al., 2006, Anal. Biochem. 358(2):281-288; Cannon et al., 2004, Anal. Biochem. 330(1):98-113; Nordin et al., 2005, Anal. Biochem. 340(2):359-368; Papalia et al., 2006, Anal. Biochem. 359(1):94-105; Stenlund et al., 2006, Anal. Biochem. 353(2):217-225; Wear et al., 2005, Anal. Biochem. 345(2):214-226). The molecules identified from the initial HTCD screen against the structure of HIV-1 MA were all in the range of 390 to 470 Da. As such, no issues are anticipated for the detection of interactions of the identified small molecules with HIV-1 Matrix. Should detection become a problem, other biophysical methods may be employed to determine affinity, such as isothermal titration calorimetry. Furthermore, direct attachment method of the HIV-1 CA protein to the sensor surface may result in denaturation of the small-molecule binding site. In a non-limiting approach to overcome this possible issue, a capture strategy may be used: biotinylated HIV-1 CA protein is attached to the surface of a streptavidin-coated sensor chip. This oriented attachment should circumvent potential problems associated with the random immobilization afforded by the amine coupling strategy.

### NMR and Crystallographic Determination of the Structure of Inhibitor-CA Protein Complexes:

Development of HSB-identified compounds may be facilitated by determining their structure in complex with the CA protein. Structural analysis may reveal elements that can be exploited to improve binding affinity and offer insights into their mechanism of action. NMR spectroscopy and X-ray crystallography studies may be used to determine the structures of the inhibitor-CA protein complexes (Kelly et al., 2007, J. Mol. Biol. 373(2):355-366; Pornillos et al., 2009, Cell 137(7):1282-1292).

### Methods of the Invention

The invention includes a method of inhibiting, suppressing or preventing an HIV-1 infection in a subject in need thereof. The method comprises administering to the subject a composition comprising a therapeutically effective amount of at least one compound selected from the group consisting of:

(a) a compound of Formula (I):

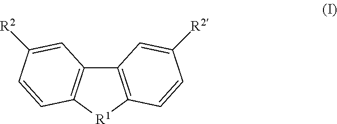

wherein in Formula (I):

R¹ is O, S, NH, N-alkyl, N—C(O)NH₂, N—CH₂C(O)NH₂, N—CH₂CH₂C(O)NH₂, CH₂, CH-alkyl, CH-OMe, CH-OEt, CH—C(O)NH₂, CH—CH₂C(O)NH₂, or CH—CH₂CH₂C(O)NH₂;

R² and R^{2′} are independently H or

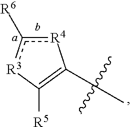

wherein,

(i) if ‘a’ is a double bond and ‘b’ is a single bond, then R³ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or

(ii) if ‘a’ is a single bond and ‘b’ is a double bond, then R³ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂; with the proviso that if R² is H then R^{2′} is not H; and

R⁵ and R⁶ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl;

(b) a compound of Formula (II):

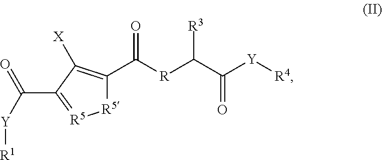

wherein:

R is NR₂, CHR₂, O or S;

R¹, R², R³ and R⁴ are independently H, alkyl, substituted alkyl, cycloalkyl, substituted cycloalkyl, aryl, substituted aryl, benzyl, substituted benzyl, heteroaryl, or substituted heteroaryl;

R⁵ is N or CH;

R^{5′} is CH₂, NH, S or O;

X is —NH₂, —NHR¹, —NR¹R², —OH, cyano, alkyl, alkoxy, halogen, sulfonamide, aryl, substituted aryl, heteroaryl or substituted heteroaryl; and,

each occurrence of Y is independently NH, NR¹, O, CH₂, CHR¹ or CR¹R²;

(c) a compound of Formula (III):

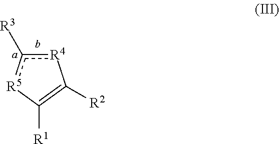

wherein in Formula (III):

R¹, R² and R³ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl,

R⁴ and R⁵ are such that:

(i) if ‘a’ is a double bond and ‘b’ is a single bond, then R⁵ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or

(ii) if ‘a’ is a single bond and ‘b’ is a double bond, then R⁵ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂;

a mixture thereof and a pharmaceutically acceptable salt thereof.

In one embodiment, the compound of Formula (I) is a compound of Formula (Ia):

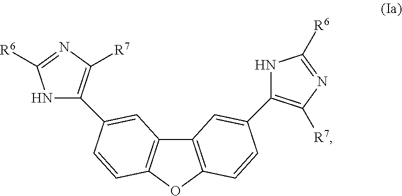

wherein in Formula (Ia):

R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

In one embodiment, the compound of Formula (I) is a compound of Formula (Ib):

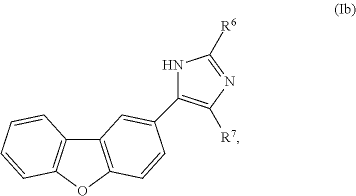

wherein in Formula (Ib):

R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

In one embodiment, the compound of Formula (III) R⁴ and R⁵ are such that:

(i) if ‘a’ is a double bond and ‘b’ is a single bond, then R⁵ is N, and R⁴ is NH or N-alkyl, or

(ii) if ‘a’ is a single bond and ‘b’ is a double bond, then R⁵ is NH or N-alkyl, and R⁴ is N.

In one embodiment, the compound is selected from the group consisting of 4,4′-(5,5′-(dibenzo[b,d]furan-2,8-diyl)bis(4-phenyl-1H-imidazole-5,2-diyl))dibenzoic acid (CMPD-A), dimethyl 4,4′-(5,5′-(dibenzo[b,d]furan-2,8-diyl)bis(4-phenyl-1H-imidazole-5,2-diyl))dibenzoate (CMPD-B), 4-(5-(dibenzo[b,d]furan-2-yl)-4-phenyl-1H-imidazol-2-yl)benzoic acid (CMPD-C), 4-amino-N⁵-[(2-chlorophenyl)methyl]-N³-cyclohexyl-N⁵-[2-(cyclohexylamino)-1-(5-methylfuran-2-yl)-2-oxoethyl]-1,2-thiazole-3,5-dicarboxamide (CMPD-D), 4-(4,5-diphenyl-1H-imidazol-2-yl)benzoic acid (CMPD-E), 4-amino-N5-benzyl-N5-(2-(benzylamino)-1-(5-methylfuran-2-yl)-2-oxoethyl)isothiazole-3,5-dicarboxamide (CMPD-G), 4-amino-N5-benzyl-N5-(2-((4-fluorobenzyl)amino)-1-(5-methylfuran-2-yl)-2-oxoethyl)isothiazole-3,5-dicarboxamide (CMPD-H), 4-amino-N5-(2-chlorobenzyl)-N5-(2-(cyclopentylamino)-1-(furan-2-yl)-2-oxoethyl)isothiazole-3,5-dicarboxamide (CMPD-J), 4-amino-N5-(2-chlorobenzyl)-N5-(2-(cyclohexylamino)-1-(5-methyl-furan-2-yl)-2-oxoethyl)isothiazole-3,5-dicarboxamide (CMPD-K), a mixture thereof, and a salt thereof.

In one embodiment, the composition further comprises one or more anti-HIV drugs. In another embodiment, the one or more anti-HIV drugs are selected from the group consisting of HIV combination drugs, entry and fusion inhibitors, integrase inhibitors, non-nucleoside reverse transcriptase inhibitors, nucleoside reverse transcriptase inhibitors, and protease inhibitors. In yet another embodiment, the subject is a mammal. In yet another embodiment, the subject is human.

The invention also includes a method of inhibiting, suppressing or preventing a viral infection in a subject in need thereof. The viral infection comprises dengue fever, dengue hemorrhagic fever, dengue shock syndrome, West Nile virus infection, or respiratory syncytial virus infection. The method comprises administering to the subject a composition comprising a therapeutically effective amount of at least one compound of Formula (I):

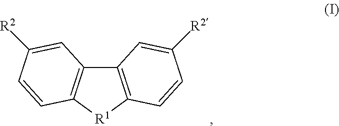

wherein in Formula (I):

R¹ is O, S, NH, N-alkyl, N—C(O)NH₂, N—CH₂C(O)NH₂, N—CH₂CH₂C(O)NH₂, CH₂, CH-alkyl, CH-OMe, CH-OEt, CH—C(O)NH₂, CH—CH₂C(O)NH₂, or CH—CH₂CH₂C(O)NH₂;

R² and R^{2′} are independently H or

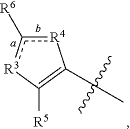

wherein,
(i) if ‘a’ is a double bond and ‘b’ is a single bond, then R³ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or
(ii) if ‘a’ is a single bond and ‘b’ is a double bond, then R³ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂;
   with the proviso that if R² is H then R^{2′} is not H; and

R⁵ and R⁶ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl, or a salt thereof.

In one embodiment, the compound of Formula (I) is a compound of Formula (Ia):

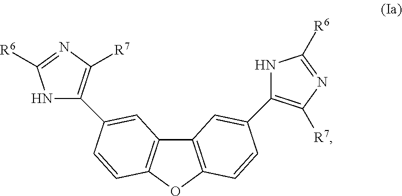

wherein in Formula (Ia):

R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

In one embodiment, the compound is selected from the group consisting of 4,4′-(5,5′-(dibenzo[b,d]furan-2,8-diyl)bis(4-phenyl-1H-imidazole-5,2-diyl))dibenzoic acid (CMPD-A), dimethyl 4,4′-(5,5′-(dibenzo[b,d]furan-2,8-diyl)bis(4-phenyl-1H-imidazole-5,2-diyl))dibenzoate (CMPD-B), a mixture thereof, and a salt thereof.

### Combination Therapies

The compounds identified using the methods described here are useful in the methods of the invention in combination with one or more additional compounds useful for treating HIV infections. These additional compounds may comprise compounds identified herein or compounds, e.g., commercially available compounds, known to treat, prevent, or reduce the symptoms of HIV infections.

In non-limiting examples, the compounds useful within the invention may be used in combination with one or more of the following anti-HIV drugs:

HIV Combination Drugs: efavirenz, emtricitabine or tenofovir disoproxil fumarate (Atripla®/BMS, Gilead); lamivudine or zidovudine (Combivir®/GSK); abacavir or lamivudine (Epzicom®/GSK); abacavir, lamivudine or zidovudine (Trizivir®/GSK); emtricitabine, tenofovir disoproxil fumarate (Truvada®/Gilead).

Entry and Fusion Inhibitors: maraviroc (Celsentri®, Selzentry®/Pfizer); pentafuside or enfuvirtide (Fuzeon®/Roche, Trimeris).

Integrase Inhibitors: raltegravir or MK-0518 (Isentress®/Merck).

Non-Nucleoside Reverse Transcriptase Inhibitors: delavirdine mesylate or delavirdine (Rescriptor®/Pfizer); nevirapine (Viramune®/Boehringer Ingelheim); stocrin or efavirenz (Sustiva®/BMS); etravirine (Intelence®/Tibotec).

Nucleoside Reverse Transcriptase Inhibitors: lamivudine or 3TC (Epivir®/GSK); FTC, emtricitabina or coviracil (Emtriva®/Gilead); abacavir (Ziagen®/GSK); zidovudina, ZDV, azidothymidine or AZT (Retrovir®/GSK); ddI, dideoxyinosine or didanosine (Videx®/BMS); abacavir sulfate plus lamivudine (Epzicom®/GSK); stavudine, d4T, or estavudina (Zerit®/BMS); tenofovir, PMPA prodrug, or tenofovir disoproxil fumarate (Viread®/Gilead).

Protease Inhibitors: amprenavir (Agenerase®/GSK, Vertex); atazanavir (Reyataz®/BMS); tipranavir (Aptivus®/Boehringer Ingelheim); darunavir (Prezist®/Tibotec); fosamprenavir (Telzir®, Lexiva®/GSK, Vertex); indinavir sulfate (Crixivan®/Merck); saquinavir mesylate (Invirase®/Roche); lopinavir or ritonavir (Kaletra®/Abbott); nelfinavir mesylate (Viracept®/Pfizer); ritonavir (Norvir®/Abbott).

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

Routes of administration of any of the compositions of the invention include oral, nasal, rectal, intravaginal, parenteral, buccal, sublingual or topical.

The regimen of administration may affect what constitutes an effective amount. The therapeutic formulations may be administered to the subject either prior to or after the onset of a viral infection. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat a viral infection in the subject. An effective amount of the therapeutic compound necessary to achieve a therapeutic effect may vary according to factors such as the state of the disease or disorder in the subject; the age, sex, and weight of the subject; and the ability of the therapeutic compound to treat a viral infection in the subject. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound useful within the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level will depend upon a variety of factors, including the activity of the particular compound employed, the time of administration, the rate of excretion of the compound, the duration of the treatment, other drugs, compounds or materials used in combination with the compound, the age, sex, weight, condition, general health and prior medical history of the subject being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds useful within the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on the unique characteristics of the therapeutic compound and the particular therapeutic effect to be achieved, and the limitations inherent in the art of compounding/formulating such a therapeutic compound for the treatment of an HIV-1 infection in a subject.

In one embodiment, the compositions of the invention are formulated using one or more pharmaceutically acceptable excipients or carriers. In one embodiment, the pharmaceutical compositions of the invention comprise a therapeutically effective amount of a compound useful within the invention and a pharmaceutically acceptable carrier.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin. In one embodiment, the pharmaceutically acceptable carrier is not DMSO alone.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from individual to individual depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds useful within the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound useful within the invention is from about 1 mg and about 2,500 mg. In some embodiments, a dose of a compound useful within the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., an HIV-1 antiviral) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments therebetween.

In one embodiment, the present invention is directed to a packaged pharmaceutical composition comprising a container holding a therapeutically effective amount of a compound useful within the invention, alone or in combination with a second pharmaceutical agent; and instructions for using the compound to treat, prevent, or reduce one or more symptoms of an HIV-1 infection in a subject.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the invention, and a further layer providing for the immediate release of a medication for HIV-1 infection. Using a wax/pH-sensitive polymer mix, a gastric insoluble composition may be obtained in which the active ingredient is entrapped, ensuring its delayed release.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents. For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing an HIV-1 infection in a subject.

The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration:

For oral administration, the compositions of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration:

For parenteral administration, the compositions of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms:

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 2003/0147952, 2003/0104062, 2003/0104053, 2003/0044466, 2003/0039688, and 2002/0051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing:

The therapeutically effective amount or dose of a compound of the present invention will depend on the age, sex and weight of the subject, the current medical condition of the subject and the nature of the infection by an HIV-1 being treated. The skilled artisan will be able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days.

The compounds for use in the method of the invention may be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials:

Unless otherwise noted, all starting materials and resins were obtained from commercial suppliers and used without purification.

Compounds CMPD-A, CMPD-B, CMPD-C, and CMPD-E were synthesized as described below. All other chemicals were purchased from commercial suppliers.

### Synthesis of CMPD-A (Scheme 1)

A solution of bromide 1 (600 mg, 1.8 mmol), CuI (35.1 mg, 0.18 mmol), PdCl₂(Ph₃P)₂ (132.9 mg, 0.18 mmol), and Ph₃P (96.6 mg, 0.37 mmol) in tetrahydrofuran (18 mL) was first degassed with argon. Phenylacetylene (0.80 mL, 7.3 mmol) was then added, followed by Et₃N (3.8 mL, 27.3 mmol). The reaction mixture was heated to 50° C. for 6 h, another 4.0 equiv. of phenylacetylene (0.80 mL, 7.3 mmol) was added, and the mixture was further stirred at this temperature overnight. The resulting mixture was cooled to room temperature, filtered, and concentrated. The crude product was passed through a pad of silica gel (20:1 hexanes/EtOAc) to give the crude alkyne 2 as an orange solid (705.9 mg), which was used directly in the next step.

To a suspension of crude alkyne 2 (705.9 mg from last step) in MeCN/H₂O (37 mL/7.4 mL) was added N-iodosuccinimide (NIS; 1.7 g, 7.6 mmol) in one portion. The reaction mixture was then heated to 75° C. for 6 h, during which time another 4.0 equiv. of NIS (1.7 g, 7.6 mmol) was added in two portions. The reaction mixture was cooled to room temperature and the resulting purple solution was diluted with EtOAc (30 mL), washed with 10% Na₂S₂O₃ (30 mL), washed with brine, dried over Na₂SO₄, and concentrated. Column chromatography purification (8:1 hexanes/EtOAc to 5:1 hexanes/EtOAc) then afforded diketone 3 as a yellow solid (421.2 mg, 53% two steps).

A suspension of diketone 3 (421.2 mg, 0.97 mmol), 4-carboxy-benzaldehyde (292.5 mg, 1.9 mmol), and NH₄OAc (2.3 g, 29.8 mmol) in AcOH (19 mL) was refluxed overnight and then cooled to room temperature. Water (10.0 mL) was then added and the solid was collected by vacuum filtration. The crude product was purified by preparative high-performance liquid chromatography (HPLC) to give the desired carboxylic acid CMPD-A as a slightly yellow solid.

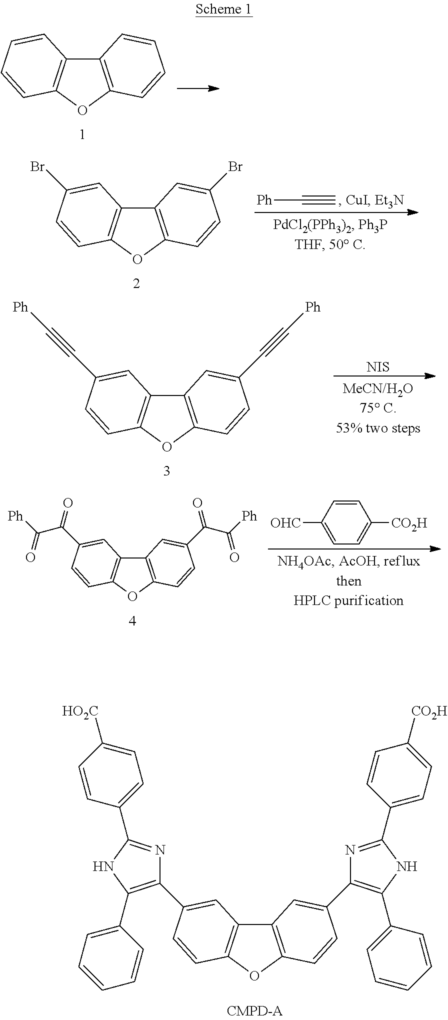

### Synthesis of CMPD-B (Scheme 2):

To a solution of diacid CMPD-A (10 mg, 14 mmol) in methanol/methylene chloride (1 μL, 1:1), approximately 50 μL of a 1.0 M solution of trimethylsilyl-diazomethane was added at room temperature until the solution became yellow and gas was no longer evolved. The reaction was then stirred for an additional 10 min. Argon was then bubbled through the reaction mixture, and the solution was diluted with additional methylene chloride. The reaction was quenched with saturated NaHCO₃. The product was extracted from the aqueous layer with methylene chloride (2×2 mL). The combined extracts were dried with Na₂SO₄ and concentrated and purified by preparative thin-layer chromatography using 1:1 hexane/ethyl acetate to yield 8.6 mg of CMPD-B (85%).

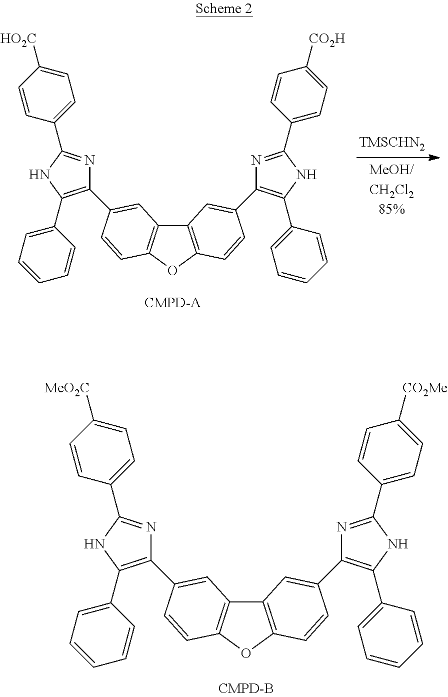 [SMILES: O=C(O)c1ccc(-c2nc(-c3ccc4oc5ccc(-c6nc(-c7ccc(C(=O)O)cc7)[nH]c6-c6ccccc6)cc5c4c3)c(-c3ccccc3)[nH]2)cc1 ; COC(=O)c1ccc(-c2nc(-c3ccc4oc5ccc(-c6nc(-c7ccc(C(=O)OC)cc7)[nH]c6-c6ccccc6)cc5c4c3)c(-c3ccccc3)[nH]2)cc1]

### Synthesis of CMPD-C (Scheme 3):

A solution of bromide 1 (200 mg, 0.81 mmol), CuI (15.4 mg, 0.081 mmol), PdCl₂(Ph₃P)₂ (56.8 mg, 0.081 mmol), and Ph₃P (42.5 mg, 0.16 mmol) in THF (8.0 mL) was first degassed with argon. Phenylacetylene (0.12 mL, 1.1 mmol) was then added, followed by Et₃N (1.7 mL, 12.2 mmol). The reaction mixture was heated to 45° C. for 4 h, another 1.3 equiv. of phenylacetylene (0.12 mL, 1.1 mmol) was added, and the mixture was further stirred for 16 h. The resulting mixture was then cooled to room temperature, filtered, and concentrated. The crude product was purified via column chromatography (pure hexanes to 1% EtOAc in hexanes) to give alkyne 2 as an orange solid (235.5 mg), which was used directly in the next step.

To a suspension of 2 (235.5 mg, 0.88 mmol) in MeCN/H₂O (9.0 mL/0.9 mL) was added NIS (592.4 mg, 2.6 mmol) in one portion. The reaction mixture was then heated to 70° C. for 3.5 h and cooled to room temperature. The resulting purple solution was diluted with EtOAc (15 mL), washed with 10% Na₂S₂O₃ (20 mL), washed with brine, dried over Na₂SO₄, and concentrated. Column chromatography purification (15:1 hexanes/EtOAc) then afforded diketone 3 as an orange solid (102.6 mg, 42% two steps).

A suspension of diketone 3 (102.6 mg, 0.34 mmol), 4-carboxy-benzaldehyde (51.3 mg, 0.34 mmol), and NH₄OAc (395.0 mg, 5.1 mmol) in AcOH (3.4 mL) was refluxed for 4 h and then cooled to room temperature. Water (5.0 mL) was then added and the solid was collected by vacuum filtration. The crude product was purified by preparative HPLC to give the desired carboxylic acid CMPD-C as a white solid.

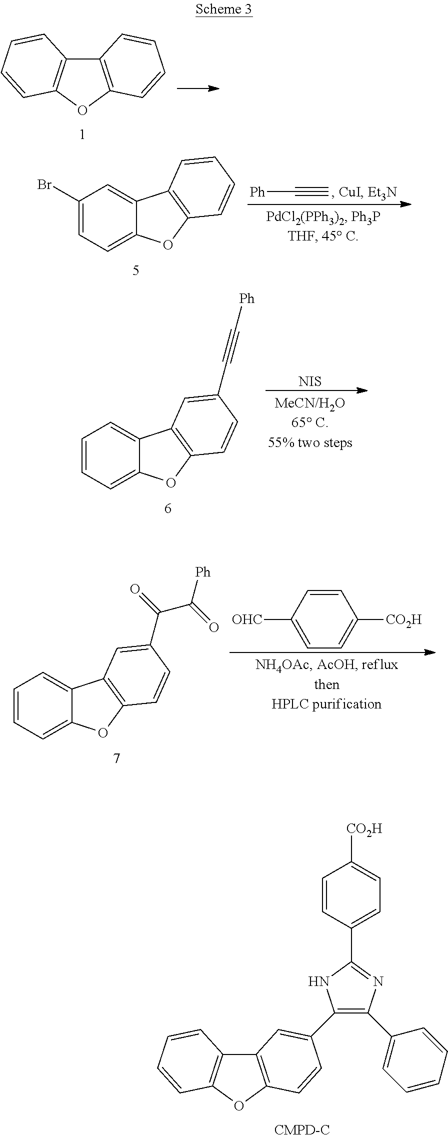

### Synthesis of CMPD-F:

Synthesis of CMPD-F was performed as detailed by Schön et al. (Schon et al., 2006, Biochemistry 45:10973-80).

### Synthesis of CMPD-E (Scheme 4):

Synthesis of CMPD-E was performed as outlined in Fridman et al. (Fridman et al., 2009, J. Mol. Struct. 917:101-09). Briefly, a mixture of benzil (400 mg, 1.9 mmol), 4-carboxybenzaldehyde (285.6 mg, 1.9 mmol), and ammonium acetate (2.2 g, 28.5 mmol) in acetic acid (19 mL) was refluxed for 4 h. The resulting mixture was then cooled to room temperature and poured into ice/water. The solid was collected by filtration and purified by column chromatography (3:2, ethyl acetate/hexanes) to yield the product as a white solid (395.3 mg, 61%).

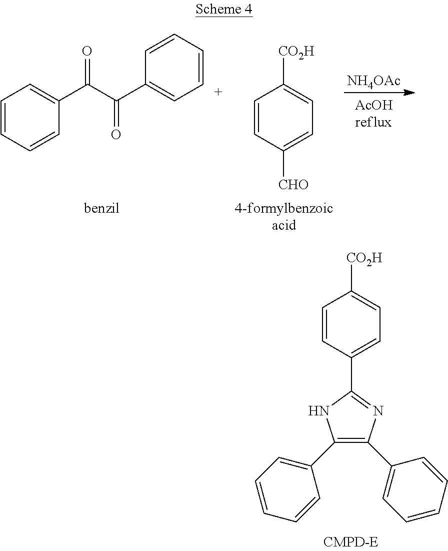 [SMILES: O=C(C(=O)c1ccccc1)c1ccccc1 ; O=Cc1ccc(C(=O)O)cc1 ; O=C(O)c1ccc(-c2nc(-c3ccccc3)c(-c3ccccc3)[nH]2)cc1 ; "NH4OAc"]

### Surface Plasmon Resonance Binding Assays:

Interaction analyses were performed on a Biacore 3000 optical biosensor (Biacore, Piscataway Township, NJ) with simultaneous monitoring of two flow cells Immobilization of the CA protein to CM7 sensor chips was performed following the standard amine coupling procedure according to the manufacturer's specifications. Briefly, carboxyl groups on the sensor chip surface were activated by injection of 50 μL of a solution containing 0.2 M EDC (1-ethyl-3-[3-dimethylamino-propyl]carbodiimide hydrochloride) and 0.05 M NHS(N-hydroxysuccinimide) at a flow rate of 5 μL min⁻¹. Next, the 50 μL of CA protein at a concentration of 6 μM in pH 5.0, 10 mM sodium acetate buffer was passed over the chip surface at 25° C. at a flow rate of 5 μL min⁻¹ Then, after unreacted protein had been washed out, excess active ester groups on the sensor surface were capped by the injection of 50 μL of 1 M ethanolamine (pH 8.0) at a flow rate of 5 μL min⁻¹. A reference surface with the nonspecific anti-gp120 antibody 17b (Thali et al., 1993, J. Virol. 67:3978-88) immobilized was generated at the same time under the same conditions and was used as background to correct nonspecific binding and for instrument and buffer artifacts.

### Direct Binding of Compounds to HIV-1 CA:

Stock solutions of CMPD-E and CMPD-F were prepared by dissolving the compounds in 100% dimethyl sulfoxide (DMSO) to a final concentration of 10 mM. To prepare the sample for analysis, 30 μL of the compound stock solution was added to sample preparation buffer (25 mM Tris-HCl, 150 mM NaCl, pH 7.5) to a final volume of 1 mL and mixed thoroughly. Preparation of analyte in this manner ensured that the concentration of DMSO was matched with that of running buffer with 3% DMSO. Lower concentrations of each compound were then prepared by twofold serial dilutions into running buffer (25 mM Tris-HCl, 150 mM NaCl, 3% DMSO, pH 7.5). These compound dilutions were then injected over the control and CA surfaces at a flow rate of 50 μL min⁻¹, for a 2-min association phase, followed by a 5-min dissociation phase. Specific regeneration of the surfaces between injections was not needed due to the nature of the interaction.

### Binding Site Analysis Via SPR.

Wild-type and mutant HIV-1 CA proteins were attached to the surface by standard amine chemistry as described above. Compound CMPD-E was injected over these surfaces at a concentration of 27.5 μM at a flow rate of 50 μL min⁻¹, for a 2-min association phase, followed by a 5-min dissociation phase, and the response at equilibrium recorded. For comparison, and to take into account minor differences in the ligand density of the mutant surfaces, responses were normalized to the theoretical Rₘₐₓ, assuming a 2:1 interaction.

### SPR Data Analysis:

Data analysis was performed using BIAEvaluation 4.0 software. The responses of a buffer injection and responses from the reference flow cell were subtracted to account for nonspecific binding. In order to obtain the equilibrium dissociation constants (K_{D}), experimental data were fitted globally to the heterogeneous ligand model. The average parameters generated from a minimum of 4 data sets were used to define the equilibrium dissociation constants (K_{D1} and K_{D2}). In the mutant CA protein studies, the average maximum response was generated from a minimum of 6 data sets and was used to define the average maximum response for compound CMPD-E binding to wild-type and mutant HIV-1 CA proteins.

### Isothermal Titration Calorimetry:

Isothermal titration calorimetric experiments were performed at 10, 15, and 25° C. using a high-precision ITC₂₀₀ titration calorimetric system from MicroCal LLC (Northampton, Mass.). All titrations were performed by adding CMPD-E in steps of 1.4 mL. All solutions contained within the calorimetric cell and injector syringe were prepared in the same buffer, 25 mM Tris-HCl, pH 7.5 with 150 mM NaCl and 3% DMSO. The concentrations of CA and CMPD-E were 35 and 600 μM, respectively. The heat evolved upon injection of CMPD-E was obtained from the integral of the calorimetric signal. The heat associated with the binding reaction was obtained by subtracting the heat of dilution from the heat of reaction. The individual heats were plotted against the molar ratio, and the values for the number of binding sites (n), the enthalpy change (ΔH) and dissociation constant (K_{D}=1/K_{A}) were obtained by nonlinear regression of the data.

### Generation of Recombinant HIV-1 Expressing Luciferase:

Using the Effectene transfection reagent (Qiagen), 293T human embryonic kidney cells were cotransfected with plasmids expressing the pCMVΔP1ΔenvpA HIV-1 Gag-Pol packaging construct, the wild-type or mutant HIV-1_{YU2} envelope glycoproteins or the envelope glycoproteins of the control amphotropic murine leukemia virus (A-MLV), and the firefly luciferase-expressing vector at a DNA ratio of 1:1:3 μg. For the production of viruses pseudotyped with the A-MLV glycoprotein, a rev-expressing plasmid was added. The single-round, replication-defective viruses in the supernatants were harvested 24-30 hours after transfection, filtered (0.45 μm), aliquoted, and frozen at −80° C. until further use. The reverse transcriptase (RT) activities of all viruses were measured as described previously (Rho et al., 1981, Virology 112:355-60).

### Assay of Virus Infectivity and Drug Sensitivity:

Cf2Th/CD4-CCR5 target cells were seeded at a density of 6×10³ cells/well in 96-well luminometer-compatible tissue culture plates (Perkin Elmer) 24 h before infection. On the day of infection, compounds of interest (1 to 100 μM) was added to recombinant viruses (10,000 reverse transcriptase units) in a final volume of 50 μL and incubated at 37° C. for 30 minutes. The medium was removed from the target cells, which were then incubated with the virus-drug mixture for 2-4 hours at 37° C. At the end of this time point, complete medium was added to a final volume of 150 μL and incubated for 48 hours at 37° C. The medium was removed from each well, and the cells were lysed with 30 μL of passive lysis buffer (Promega) by three freeze-thaw cycles. An EG&G Berthold Microplate Luminometer LB 96V was used to measure luciferase activity in each well after the addition of 100 μL of luciferin buffer (15 mM MgSO₄, 15 mM KPO₄ [pH 7.8], 1 mM ATP, 1 mM dithiothreitol) and 50 μL of 1 mM D-luciferin potassium salt (BD Pharmingen).

### Conservation Analysis:

The CA protein sequences for the various isolates were obtained either from the HIV-1 sequence repository at the bioafrica project (bioafrica.net) (de Oliveira, T., et al., 2005, Bioinformatics 21:3797-3800) or from swiss prot sequence repository (Bairoch et al., 2004, Brief Bioinform 5:39-55). The sequences were aligned using a multiple sequence alignment program (ClustalW) (Higgins et al., 1996, Methods Enzymol 266:383-402). The aligned sequences were then analyzed for evolutionary and functional conservation using the ConSurf algorithm (Ashkenazy et al., 2010, Nucleic Acids Res 38:W529-533) and were mapped onto the crystal structure of the CA monomer of the HIV-1NL4-3 isolate. In addition, the three dimensional structure of these isolates were modeled using Modeler (version 9.4) with the crystal structure monomer (derived from 3H4E (Pornillos et al., 2009, Cell 137:1282-92)) as the template. The resulting models were energy minimized using Amber charges and Amber force field adopted in MOE. Further, the models were subject to normal mode analysis as described previously. CMPD-A, CMPD-B, CMPD-C, and CMPD-E were docked to the monomeric interface region using GOLD docking program and scored using goldscore and chemscore.

Overproduction and Purification of his-Tagged Wild-Type and Mutant HIV-1 CA:

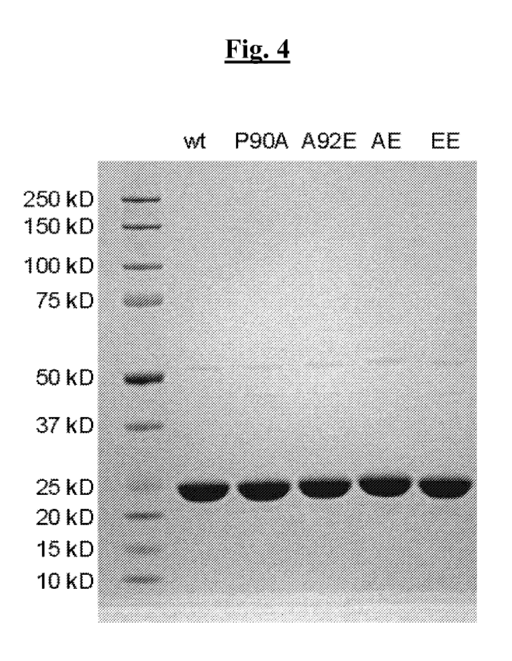

Bacterial expression plasmids that afford high-level overproduction of wild-type and mutant His-tagged HIV-1 CA proteins were used (Li et al., 2009, J. Virol. 83(21):10951-10962). The double mutants P90A/A92 and P90E/A92E were herein designated as AE and EE. His-CA proteins were overproduced in BL21(DE3) cells (novagen) upon induction by treatment with 1 mM IPTG (isopropyl-β-D-thiogalactopyranoside; Sigma). Cells were harvested 4 h after induction, the soluble CA protein was extracted by disruption by sonication and purified by immobilized metal affinity chromatography (IMAC) using Ni-NTA resin (Qiagen, Germantown, Md.), yielding large amounts of pure protein. In the SDS-PAGE displayed in FIG. 4, each band corresponds to twenty micrograms of a purified CA protein stained with Coomassie blue.

In one example, two milliliters of LB, containing 100 μg mL⁻¹ ampicillin and 50 μg mL⁻¹ chloramphenicol, were inoculated with a single transformed colony and allowed to grow at 37° C. for 9 h. A total of 100 μL of the preculture was used to inoculate 100 mL of the autoinducing media ZYP-5052 (Studier, 2005, Protein Expr. Purif. 41:207-34) containing 100 μg mL⁻¹ ampicillin and 34 μg mL⁻¹ chloramphenicol. The culture was grown at 30° C. for 16 h. Cells were harvested by centrifugation at 1076×g for 20 min at 4° C. and the pellet was suspended in 30 mL phosphate-buffered saline (PBS; Roche, Pleasanton, Calif.) containing 2.5 mM imidazole. Cells were lysed by sonication and the supernatant clarified by centrifugation at 11,952×g (SS-34, Sorvall RC 5C Plus; DuPont, Wilmington, Del.) for 20 min at 4° C. The supernatant was removed and applied to a TALON cobalt resin affinity column (ClonTech Laboratories, Mountain View, Calif.), previously equilibrated with PBS, 2.5 mM imidazole. Loosely bound proteins were removed via seven-column volumes of PBS containing 7.5 mM imidazole. Tightly associated proteins were eluted in three-column volumes of PBS containing 250 mM imidazole. The eluates were then pooled and then dialyzed at 4° C. overnight against 2 L of 20 mM Tris-HCl, pH 8.0, concentrated to 120 μM, flash frozen in liquid nitrogen, and stored at −80° C. until further use. Mutant CA proteins were purified as described above for the wild-type CA protein.

### In Vitro Assembly of HIV-1 CA:

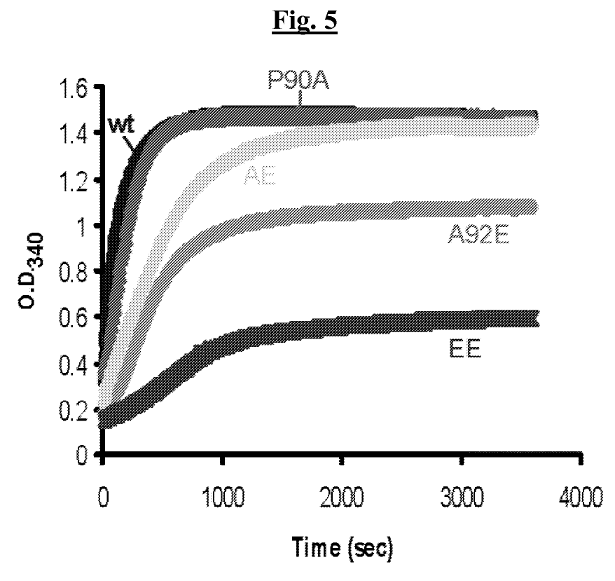

Soluble HIV-1 CA protein may be triggered to assemble into tubes similar in diameter and morphology to intact cores by dilution into high-ionic-strength buffer. The kinetics of assembly of wild-type and mutant HIV-1 CA protein was followed by monitoring the increase in turbidity using a spectrophotometer (Li et al., 2009, J. Virol. 83(21):10951-10962). The curves displayed in FIG. 5 illustrated the fact that mutations in CA protein cause differences in assembly kinetics. In FIG. 5, each CA protein, at a concentration of 44 μM, was assembled in 2.5 M NaCl. The optical density at 340 nm was monitored every 10 seconds over a time period of one hour.

In vitro CA Assembly Assay:

The effect of compound CMPD-E on the assembly of HIV-1 CA was measured by monitoring turbidity at 350 nm using a modification of the method of Tian et al. (Tian et al., 2009, Bioorg. Med. Chem. Lett. 19:2162-67). Briefly, 1.0 μL of concentrated CMPD-E in 100% DMSO was added to a 74-μL aqueous solution (2 mL of 5 M NaCl mixed with 1 mL of 200 mM NaH₂PO₄, pH 8.0). To initiate the assembly reaction, 25 μL of purified CA protein (120 μM) was added. An identical reaction mixture was prepared, omitting the compound (i.e., DMSO only). Samples were allowed to equilibrate for 2 min prior to reading. Readings were taken at 350 nm every 10 s for 30 min. CA was used at a final concentration of 30 μM, and CMPD-E at a final concentration of 147 μM.

### Anti-HIV Efficacy Evaluation in MAGI Cell Lines:

P4-R5 MAGI cells (NIH AIDS Research & Reference Reagent Program, catalog #3580) were maintained in Dulbecco's Modified Eagle's Media (DMEM) supplemented with 10% fetal bovine serum (FBS), sodium bicarbonate (0.05%), antibiotics (penicillin, streptomycin, and kanamycin at 40 μg/mL each), and puromycin (1 μg/mL). Initial studies were performed using the HIV-1-susceptible P4-R5 MAGI reporter cell line. HIV-1 infection of these cells (which express CD4, CXCR4, and CCR5) results in LTR-directed β-galactosidase expression, which can be readily and accurately quantitated. Approximately 18 h prior to the experiment, P4-R5 MAGI cells were plated at a concentration of 1.2×10⁴ cells/well in a flat-bottom 96-well plate. On the day of the experiment, cells were infected in quadruplicate with HIV-1 strain IIIB (Advanced Biotechnologies, Inc., Columbia, Md.) in the presence or absence of putative CA inhibitors at the indicated concentrations. After 48 h incubation at 37° C., cells were assayed for infection using the Galacto-Star One-Step β-Galactosidase Reporter Gene Assay System (Applied Biosystems, Bedford, Mass.). Each EC₅₀ (concentration at which exposure to the compound resulted in a 50% decrease in infection relative to mock-treated, HIV-1-infected cells) was calculated using the Forecast function of the Microsoft Excel.

### Anti-HIV Efficacy Evaluation in Human Peripheral Blood Mononuclear Cells:

HIV human peripheral blood mononuclear cell (PBMC) assays were performed as described previously (Lanier et al., 2010, Antimicrob. Agents Chemother. 54:2901-09; Ptak et al., 2008, Antimicrob. Agents Chemother. 52:1302-17). Briefly, fresh PBMCs, seronegative for HIV and hepatitis B virus (HBV), were isolated from blood samples of the screened donors (Biological Specialty Corporation, Colmar, Pa.) by using lymphocyte separation medium (LSM; density, 1.078±0.002 g/ml; Cellgro; Mediatech, Inc.) by following the manufacturer's instructions. Cells were stimulated by incubation in 4 μg/mL phytohemagglutinin (PHA; Sigma) for 48 to 72 h. Mitogenic stimulation was maintained by the addition of 20 U/mL recombinant human interleukin-2 (rhIL-2; R&D Systems, Inc.) to the culture medium. PHA-stimulated PBMCs from at least two donors were pooled, diluted in fresh medium, and added to 96-well plates at 5×10⁴ cells/well. Cells were infected (final multiplicity of infection [MOI] of ≅0.1) in the presence of 9 different concentrations of test compounds (triplicate wells/concentration) and incubated for 7 days. To determine the level of virus inhibition, cell-free supernatant samples were collected for analysis of reverse transcriptase activity (Buckheit Jr., et al., 1991, AIDS Res. Hum. Retroviruses 7:295-302). Following removal of supernatant samples, compound cytotoxicity was measured by the addition of 3-(4,5-dimethylthiazol-2-yl)-5-(3-carboxymethoxyphenyl)-2-(4-sulfophenyl)-2H-tetrazolium (MTS; CellTiter 96 reagent; Promega) by following the manufacturer's instructions.

Virus isolates were obtained from the NIH AIDS Research and Reference Reagent Program, Division of AIDS, NIAID, NIH, as follows: HIV-1 Group M isolates 92UG031 (Subtype A, CCR5-tropic), 92BR030 (Subtype B, CCR5-tropic), 92BR025 (Subtype C, CCR5-tropic), 92UG024 (Subtype D, CXCR4-tropic), and 93BR020 (Subtype F, CCR5/CXCR4 Dual-tropic) from the UNAIDS Network for HIV Isolation and Characterization (Gao et al., 1994, AIDS Res. Hum. Retroviruses 10:1359-68); HIV-1 Group M isolate 89BZ167 (Subtype B, CXCR4-tropic; also referred to as “89BZ_{—}167”, “89_BZ167”, “BZ167” or “GS 010”) from Dr. Nelson Michael (Brown et al., 2005, J. Virol. 79:6089-6101; Jagodzinski et al., 2000, J. Clin. Microbiol. 38:1247-49; Michael et al., 1999, J. Clin. Microbiol. 37:2557-63 and Vahey et al., 1999, J. Clin. Microbiol. 37:2533-37); HIV-1 Group M isolate 931N101 (Subtype C, CCR5-tropic) from Dr. Robert Bollinger and the UNAIDS Network for HIV Isolation and Characterization (Gao et al., 1994, AIDS Res. Hum. Retroviruses 10:1359-68); HIV-1 Group M isolate CMUO8 (Subtype E, CXCR4-tropic) from Dr. Kenrad Nelson and the UNAIDS Network for HIV Isolation and Characterization (Gao et al., 1994, AIDS Res. Hum. Retroviruses 10:1359-68); HIV-1 Group M isolate G3 (Subtype G, CCR5-tropic) from Alash'le Abimiku (Abimiku et al., 1994, Aids Res. Hum. Retrovir. 10:1581-83); HIV-1 Group 0 isolate BCF02 (CCR5-tropic) from Sentob Saragosti, Françoise Brun-Vézinet, and François Simon (Loussertajaka et al., 1995, J. Virol. 69:5640-49); and SIV isolate Mac251 from Dr. Ronald Desrosiers (Daniel et al., 1985, Science 228:1201-04).

### Determination of Antiviral Spectrum of CA-Targeted Compounds:

To determine the spectrum of antiviral activity of the hit compounds, they were evaluated for inhibition of cytopathic effect (CPE) against a panel of viruses from different classes, as described previously. In the CPE assay the test virus is pretitered such that control wells exhibit 85% to 95% loss of cell viability due to virus replication. Therefore, antiviral effect, or cytoprotection, is observed when compounds prevent virus replication. The effects of the compounds on herpes simplex virus-1 (dsDNA; strain HF evaluated in Vero cells; virus and cells obtained from the American Type Culture Collection) were assessed as described previously. The following viruses were all screened at IBT Bioservices (Gaithersburg, Md.) for compound-dependent inhibition of virus-induced cytopathic effect: Japanese encephalitis (JEV, strain 14-14-2; Nepal JEV Institute), yellow fever (YFV, strain 17-D; United States Army Medical Research Institute for Infectious Disease [USAMRIID]), Chikungunya (CHIKV, strain 181-25; USAMRIID), Dengue-2 (DENV2, strain New Guinea C; University of Texas Medical Branch [UTMB]), Dengue-1 (DENV1, strain TH-S-MAN; UTMB), Dengue-3 (DENV3, strain H87; UTMB), respiratory syncytial virus (RSV, strain A2; Functional Genetics), Vaccinia (VACCV, strain NYCBH; USAMRIID), Dengue-4 (DENV4, strain H241; UTMB), influenza H1N1 (INFV, strain A/PR/68; Charles River Labs).

Vero cells (for viruses DENV, JEV, RSV, CHIKV, and YFV), BSC-40 cells (virus VACCV), or Madin-Darby canine kidney cells (virus INFV) were seeded in 96-well plates at 10⁴ cells per well in Dulbecco's modified minimal essential medium (virus VACCV), minimal essential medium (virus DENV, JEV, RSV, CHIKV, and YFV), or UltraMDCK (virus INFV, supplemented with 1 μg/ml tosyl phenylalanyl chloromethyl ketone-treated trypsin), containing 2 mM L-glutamine, 100 units/ml penicillin, 100 ng/ml streptomycin, and FBS (Invitrogen; 5% FBS for VACCV, 1% FBS for JEV, YFV, DENV, RSV, and 0% for INFV). Cells were incubated in a humid 37° C. incubator containing 5% CO₂. Dose-response curves were generated by measuring CPE at a range of compound concentrations. Eight compound concentrations (100, 50, 25, 12.5, 6.25, 3.13, 1.56, and 0.78 μM) were used to generate inhibition curves suitable for calculating the IC₅₀ from virus-induced CPEs. Compound dilutions were prepared in DMSO prior to addition to the cell culture medium. The final DMSO concentrations in all samples were 0.1%. Cells were infected with approximately 0.1 plaque-forming units (PFU) per cell approximately 1 hour after addition of compound. At 4 to 6 days after infection (the exact duration dependent on the virus), cultures were fixed with 5% glutaraldehyde and stained with 0.1% crystal violet in 5% methanol. Virus-induced CPE was quantified spectrophotometrically by absorbance at 570 nm.

IC₅₀ values were calculated by fitting the data to a four-parameter logistic model to generate a dose-response curve using XLfit 5.2 (equation 205, IBDS, Emeryville, Calif.). The linear correlation coefficient squared (R²) for fitting data to this model was typically >0.98%. From this curve, the concentration of compound that inhibited virus-induced CPE by 50% was calculated. As controls, uninfected cells and cells receiving virus without compound were included on each assay plate, as well as the reference agent ribavirin (Sigma, St. Louis, Mo.) when applicable.

### Example 1

### Screening of a Novel Enriched Database of Small Molecules Using the HSB Method and the Structure of the HIV-1 CA Protein

An iterative in silico-in vitro method called the hybrid structure-based (HSB) method was used for screening small molecules to inhibit the CA NTD-NTD interface. The initial HSB protocol for designing small-molecule inhibitors to G-protein coupled receptors has been described in detail by Kortagere and Welsh (Kortagere et al., 2006, J. Comput. Aided Mol. Des. 20:789-802). The HSB method was recently customized to design protein-protein interaction inhibitors of Plasmodium falciparum (Bergman et al., 2007, “Small Molecule Inhibitors of the P. falciparum MyoA Tail-MTIP Intercation”, Molecular Parasitology meeting XVIII, MBL, Woods Hole; Kortagere et al., 2010, J. Chem. Inf. Model. 50:840-49). The protocol consists of multiple phases that are used in an iterative manner.

A comprehensive electronic database of commercially available small molecules was developed as the first phase of the HSB method. This database was generated using a subset of the Zinc database that consists of compounds from commercial vendors such as Asinex (Moscow, Russia), Maybridge (Trevillett, North Cornwall, UK), Bionet (Camelford, Cornwall, UK), Cerep (Paris, France), AMRI (Albany, N.Y.), and TimTec (Newark, Del.) along with other compounds from natural sources, ligands from the Protein Data Bank (PDB), and FDA-approved drugs. The entire database was comprised of nearly 3 million compounds. All of the commercially available compounds were acquired as sdf formatted files, converted into the Mol112 format, and energy minimized in SYBYL (Tripos, St. Louis, Mo.). All of the molecules in the database were filtered for redundancy and renamed according to their corresponding vendor listing.

The next phase of the HSB method was the generation of the combined ligand-protein pharmacophore (also called the hybrid pharmacophore). A model of the CA-CA complex was prepared from PDB entry 3H4E by adding hydrogen atoms and refining the structure using energy minimization combined with a 1-ns-long molecular dynamics simulation. All simulations were performed using Amber (version 9.0), with Amber charges as adopted in Molecular Operating Environment (MOE) program (version 10; Chemical Computing Group, Montreal, Quebec, Canada). Further, the flexibility of the CA interface was assessed using normal mode analysis. An elastic network model as adopted in the elNémo webserver (http://igs-server.cnrs-mrs.fr/elnemo/start.html) (Suhre et al., 2004, Nucleic Acids Res. 32:W610-614) was used to compute the models by perturbing the system along the chosen low-frequency vibrational mode. The first five low-frequency modes were considered for each model and all the resulting conformations were stored in pdb format. The models were superimposed to derive average distance and angles between the interface residues, which were then converted into flexible distance restraints for use in the pharmacophore design.

The combined pharmacophore was then designed centered around those residues responsible for the stability of the interface. Site-directed mutagenesis studies have shown that residues A42 and M39 when mutated prevent capsid assembly. These two residues along with L20 from the neighboring monomer form the hydrophobic core of the hotspot (FIG. 2B), while R173 forms a critical interdomain hydrogen bond with N57 and V59. A four-point pharmacophore consisting of three hydrophobic and one hydrogen bond donor-acceptor feature was designed using these interactions as input.

The enriched database described above was then screened against this pharmacophore and first filtered according to Lipinski's “rule of five” to identify “drug-like” molecules. A second regression-based blood-brain barrier (BBB) penetration model was also applied to filter out compounds for BBB penetration. This pharmacophore-based screening and filtering afforded 900 hits. From these 900 hits, 300 hits were selected for docking and scoring to the structure of a monomer isolated from the hexameric CA protein structure. The GOLD program (Genetic Optimisation for Ligand Docking) in “library screening mode” was employed for preliminary docking and scoring. The docking area was restricted by a sphere of 8 Å and encompassed residues from the interface region such as P38, T58, A42, M39, and L20. Given the non-deterministic nature of genetic algorithms, 50 independent docking runs were performed for each ligand. The full set of docked structures was then energy minimized using the molecular modeling package SYBYL. The docked receptor-ligand complexes were then scored using a customizable knowledge-based scoring function based on the nature of the interaction of every atom within the NTD-NTD docking pharmacophore (Kortagere & Welsh, 2006, J. Comput. Aided Mol. Des. 20(12):789-802). A consensus scoring scheme that involves GoldScore, ChemScore, contact score, and a shape-weighted scoring scheme (Kortagere et al., 2009, Pharm. Res. 26(4):1001-1011) was then used to rank the compounds. The best ranking complexes were visually inspected to include compounds that not only interacted with the specified residues but also had extended volume to maximize the inhibition of the NTD-NTD interface.

### Example 2

### Identification of CMPD-A as an Early-Stage Inhibitor of HIV-1 Replication

The HSB method (Kortagere et al., 2009, Pharm. Res. 26:1001-11; Kortagere et al., 2010, Environ. Health Perspect. 118(10):1412-17; Kortagere et al., 2006, J. Comput. Aided Mol. Des. 20:789-802; Kortagere et al., 2010, J. Chem. Inf. Model 50:840-49 and Peng et al., 2009, Bioorg. Med. Chem. 17:6442-50) was used to design small-molecule inhibitors targeted to the NTD-NTD hexameric interface of HIV-1 CA. The HSB method, as the name implies, is a hybrid method combining elements of ligand-based and structure-based virtual screening strategies: using ligand-based methods to build enriched libraries of small molecules, and then employing a combined receptor-ligand pharmacophore to screen molecules from the enriched library and to further dock the molecules to their receptor. The docked complexes are then scored based on a number of physicochemical parameters to indicate high-ranking molecules. The results of this detailed analysis of the dynamic mode of association between the receptor and ligand are then used to list candidate molecules that are suitable for biological and biochemical testing. Screening with the hybrid pharmacophore resulted in 900 hits that were filtered for drug-like properties. The molecules were also screened using principal component analysis to identify those with unique chemical cores, which resulted in ˜300 hits. These molecules were then docked into the dimeric interface region of the CA monomer and scored using a goldscore, chemscore and a customized scoring scheme. From the 300 docked complexes, the 25 best ranking molecules were purchased for analysis of antiviral effect using single-round infection assays. Details of the single-round infection assay have been published in detail elsewhere and the method has been routinely used for phenotypic characterization of HIV-1 envelope glycoproteins and studies of inhibitors of HIV-1 replication (Madani et al., 2007, J. Virol. 81:532-38; Madani et al., 2008, Structure 16:1689-1701; Si et al., 2004. Proc. Natl. Acad. Sci. USA 101:5036-41). Effects on early-stage events by the compounds were determined by producing virus in the absence of compound, then exposing target cells to virus in the absence or presence of various concentrations of compounds.

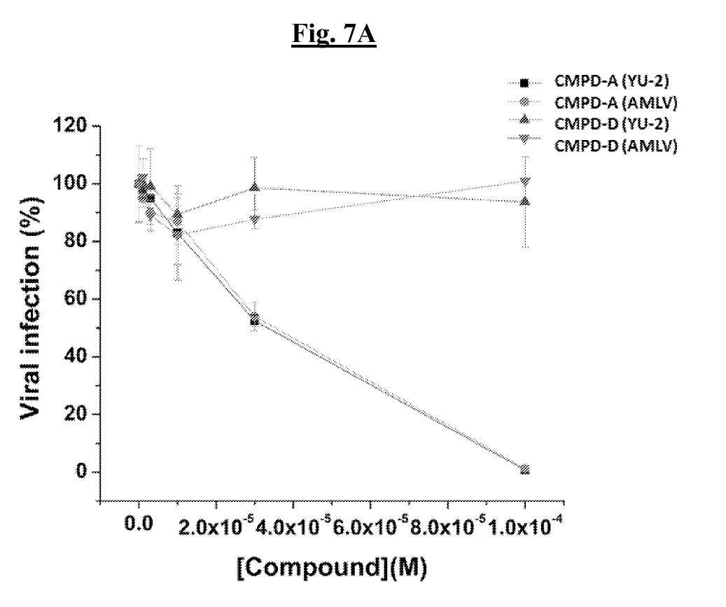

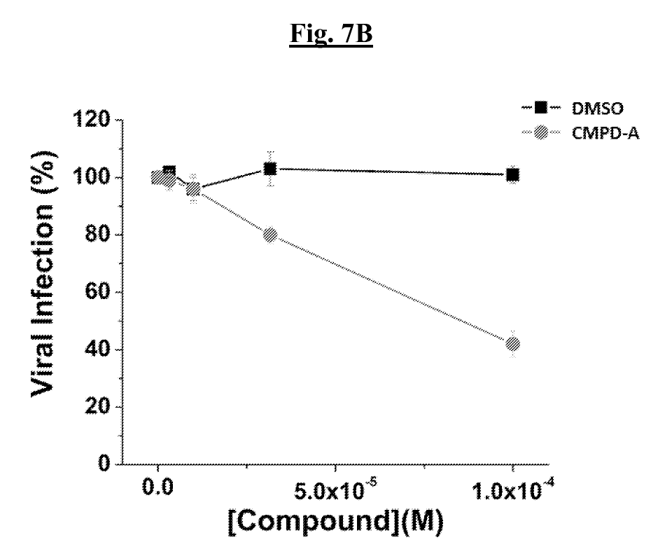

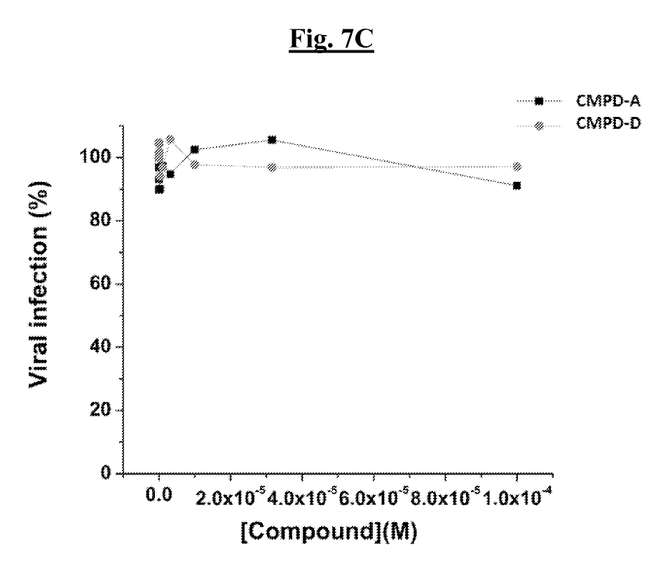

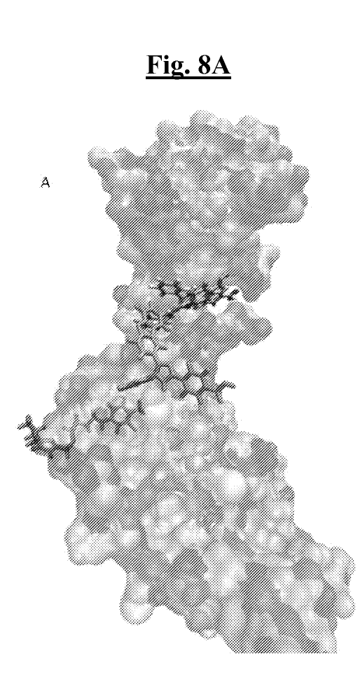

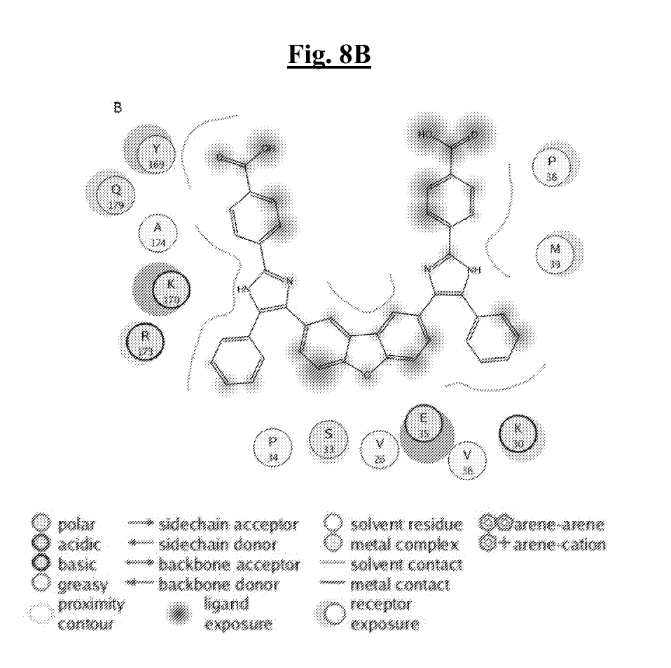

From this initial screen, one compound 4,4′-[dibenzo[b,d]furan-2,8-diylbis(5-phenyl-1H-imidazole-4,2-diyl)]dibenzoic acid, referred to as CMPD-A, was identified as having anti-HIV activity of 33.3±0.31 μM on the infection of recombinant luciferase-containing HIV-1 viruses (HIV-1_{NL4-3} backbone) pseudotyped with the envelope protein from HIV-1_{YU-2}. CMPD-A was found to disrupt infection at an early, post-entry stage (FIG. 7A) as its activity was independent of Env-mediated fusion, inhibiting HIV-1 pseudotyped with the envelope glycoprotein from murine leukemia virus (MLV). Although production of pseudovirions by transfection and the ability to analyze inhibition in a single-round infection are advantageous for addressing the inhibitory effect of a given compound, this type of assay cannot address the effects of multiple rounds of infection and cell-to-cell spread on the efficacy of the test compounds. CMPD-A was evaluated for inhibition of replication of fully infections virus (FIG. 8B). The compound was assessed against fully infectious HIV-1_{IIIB} replicating in the P4-R5 MAGI cell line. This analysis demonstrated that CMPD-A could inhibit the replication of this isolate with an IC₅₀ of 89±3.2 μM. The P4-R5 MAGI cell line is a HeLa derivative and is therefore not a natural target cell type, only being able to support infection by HIV-1 by overexpression of CD4, CXCR4 and CCR5. Moreover, HIV-1_{IIIB} is a laboratory adapted virus, having been multiply passaged in culture, and lacks some of the accessory proteins. CMPD-A was evaluated for inhibition of a primary isolate, HIV-1_{92BR030}, replicating in primary PBMCs (FIG. 8C). Interestingly, the compound displayed no activity in the PBMC assay, despite being available and stable in the media over the course of the experiment.

### Example 3

### Size Reduction and Optimization of CMPD-A: Identification of CMPD-E

Compound CMPD-A displayed activity in single- and multiple-round infection assays using cell lines, but was unable to inhibit the primary isolate HIV-1_{92BR030} replicating in PBMCs. This compound probably suffered from poor permeability across the PBMC membrane. This is probably a function of its poor drug like properties (high octonol water partition co-efficient (logP) of 9.31 as determined using the weighted logP function in JChem) and large molecular weight (692 Da). As such, attempts were made to reduce the size of the compound and optimize its physical-chemical properties, as to improve its PBMC permeability, while retaining its antiviral activity. CMPD-A has a C2 symmetry along the central dibenzofuran ring and docking results suggested that the proposed binding area of CMPD-A spans the entire NTD dimer interface including the junction between the N and C-terminal lobes (FIGS. 8A and 8B).

Based on the docking model, the upper arm of CMPD-A was proposed to interact with residues R173, D166, K170, Y169, E180, Q179, S33, and P34 while the lower arm with P38, M39, E35, K30 and V36. Due to its symmetry, docking solutions indicated that either arm could occupy either of the two sites, there was no particular preference for one arm over the other.

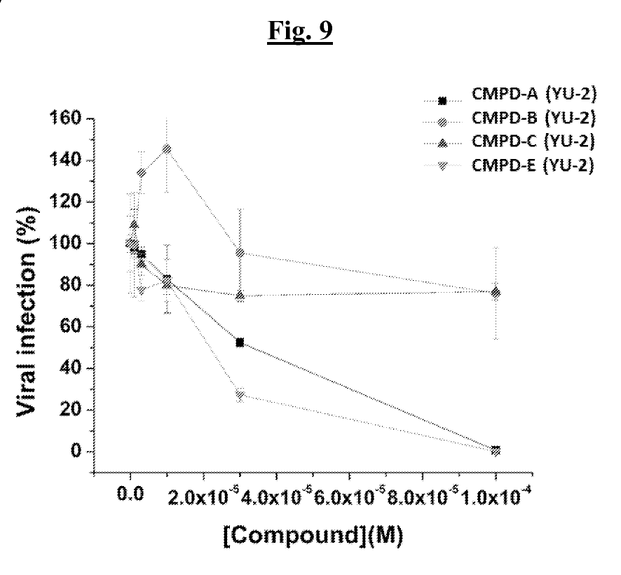

In order to test these docking observations, two analogs of CMPD-A were designed. The first, CMPD-C, is composed of the furan ring linker region attached to one arm of the parental molecule, whereas the other, CMPD-E corresponded to only the arm structure. In addition, the benzoic acid moiety on CMPD-A was predicted from the docking pose to form hydrogen bond interactions with Gln179 and Glu180 of CA. This prediction, along with the criticality of these potential interactions to the antiviral activity of CMPD-A, were tested by synthesizing CMPD-B, a dimethyl ester variant of CMPD-A, which removed the hydrogen-bonding capability at this region. These compounds were then subject to antiviral analysis using the single round infection assay. As can be seen in FIG. 9, CMPD-B lost all activity in the single-round infection assay, indicating that potential hydrogen bonds formed by benzoic acid moiety in CMPD-A are key to its activity. Compounds CMPD-E and CMPD-C, however, retained the activity of the parental molecule. CMPD-E, which retains the antiviral activity of CMPD-A, represents a significant reduction in molecular size (692 vs 340 Da) and improvement in physical-chemical properties (logP 9.31 vs 5.05), so CMPD-E was tested in the PBMC assay. As can be seen in FIG. 9, CMPD-E has a comparable IC₅₀ value for inhibition of the HIV-1_{BR030} isolate replicating in primary PBMCs as the parental CMPD-A exhibited against HIV-1_{IIIB} replicating in P4-R5 MAGI cells.

### Example 4

### CMPD-E Binds to HIV-1 CA and Stops its Assembly In Vitro

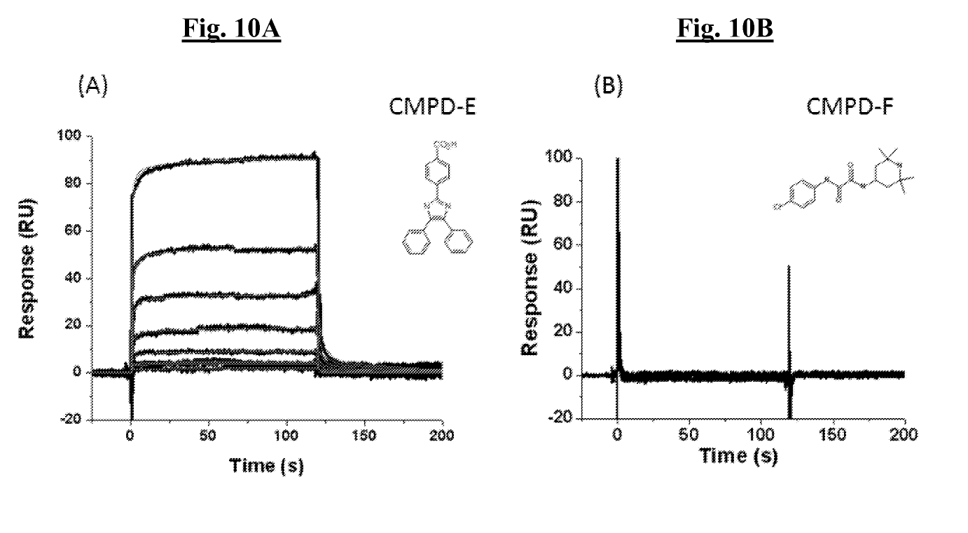

Compound CMPD-E is predicted to interact with the NTD of HIV-1 CA and thereby alter its assembly. However, it is possible that the compound exerts its action via another mechanism not involving CA. Studies were thus performed to establish that CMPD-E is directed against HIV-1 CA. The direct interaction of CMPD-E was assayed using surface plasmon resonance (SPR) interaction analyses. Wild-type HIV-1 CA protein was purified and immobilized onto the surface of a high-capacity CM7 sensor chip. A surface to which the monoclonal antibody 17b (a generous gift from Dr. James E. Robinson, Department of Pediatrics, Tulane University Medical Center, New Orleans, La.) was immobilized was used to correct for background binding and instrument and buffer artifacts. CMPD-E directly interacted with sensorchip-immobilized HIV-1 CA (FIG. 10A). Moreover, the small-molecule CD4 mimetic compound CMPD-F displayed no such interaction with HIV-1 CA, establishing the specificity of CMPD-E for HIV-1 CA (FIG. 10B). Interestingly, fitting of the SPR data indicated that the CMPD-E interacted with HIV-1 CA with a 2:1 stoichiometry. Therefore, in order to determine whether this was a real stoichiometry and not an artifact of immobilization, isothermal titration calorimetry was performed.

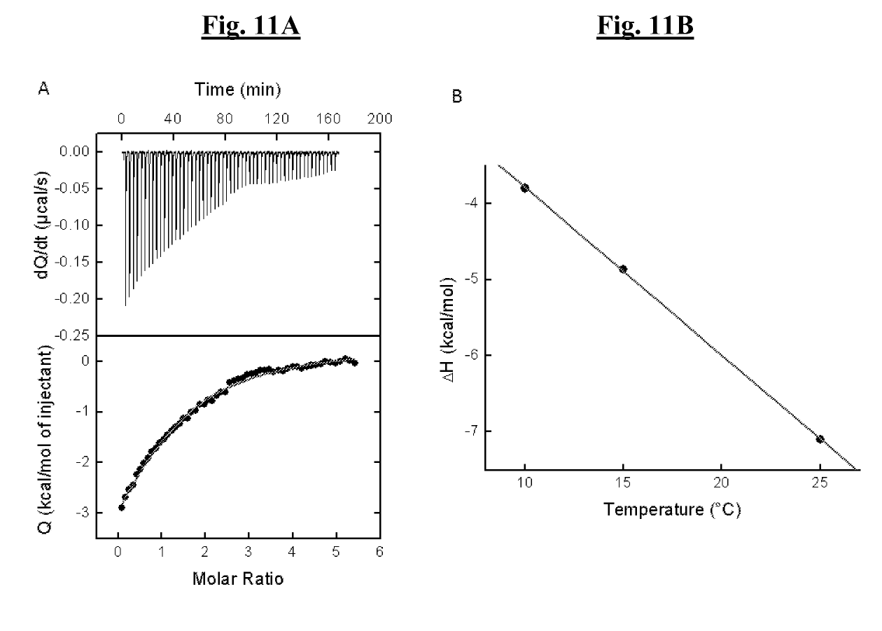

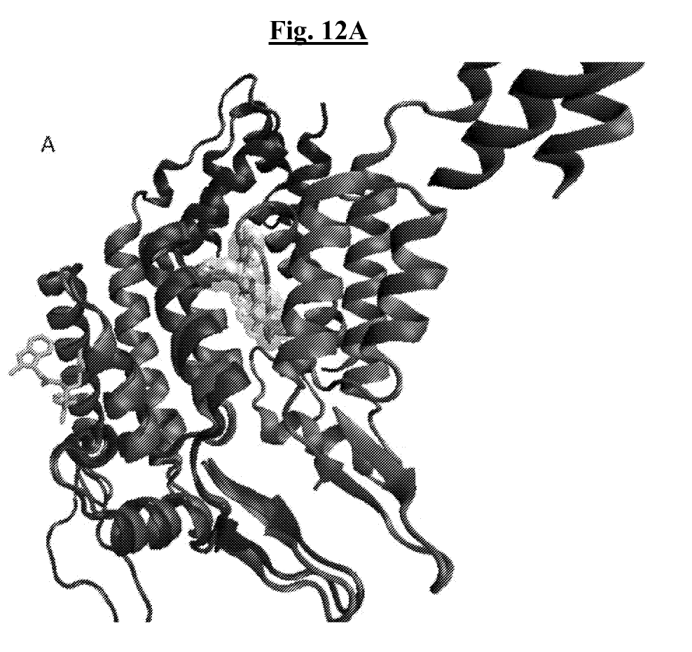

In order to determine the validity of the apparent 2:1 stoichiometry in solution, the binding of CMPD-E to CA was measured by isothermal titration calorimetry (ITC). FIG. 11A illustrates the calorimetric titration of HIV-1_{NL4-3} CA with CMPD-E at 25° C. in Tris-HCl, 150 mM NaCl with 3% DMSO (the exact buffer used for the SPR experiment). The experimental data fitted to a binding model wherein two molecules of CMPD-E bind to one CA molecule both with equal affinity. This finding therefore supports both the stoichiometry obtained from SPR analysis. The affinity of the CMPD-E—CA interaction at 25° C. was determined to be 85 μM (for both sites), corresponding to a change in Gibbs energy of −6.6 kcal/mol. The changes in enthalpy (ΔH) and entropy (ΔS) are −7.3 kcal/mol and −5.0 cal/(K×mol), respectively, and the change in heat capacity, calculated from temperature dependence of the enthalpy, is −220 cal/(K×mol) (FIG. 11B). As such, the measured thermodynamic parameters are indicative of a profile of a typical small molecule-protein interaction that binds without inducing any major conformational changes (Mobley et al., 2009, Structure 17:489-98; Ohtaka et al., 2005, Prog. Biophys. Mol. Biol 88:193-208).

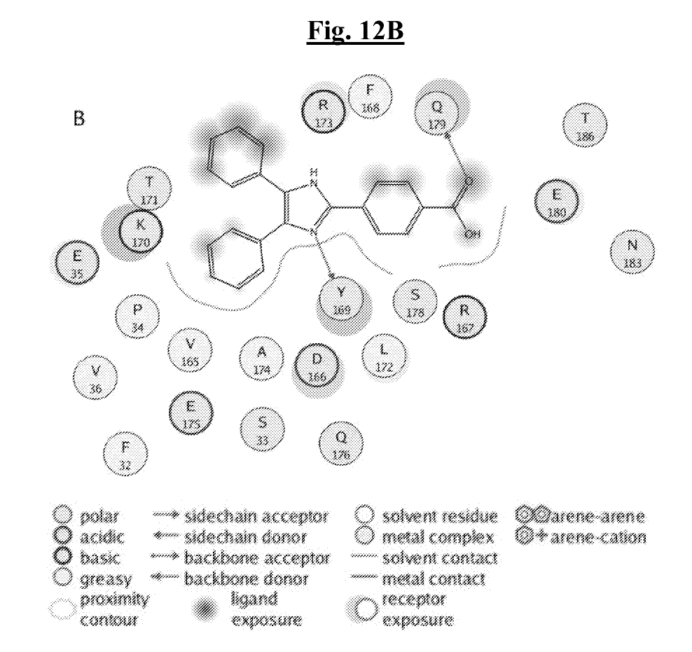

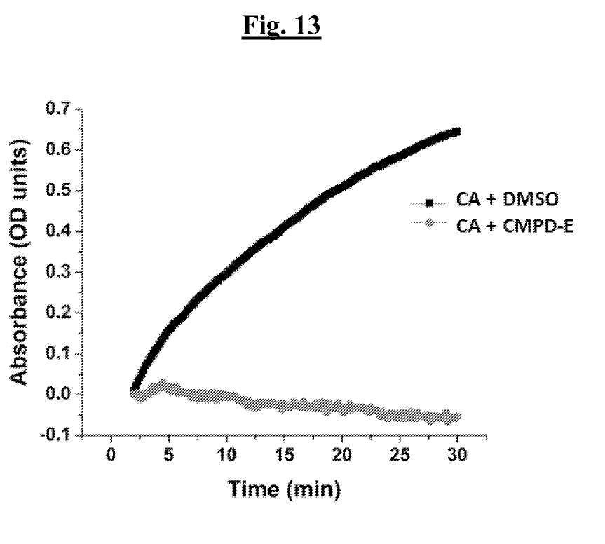

Having demonstrated that CMPD-E directly interacts with HIV-1 CA, studies were then performed to assess whether it affects the assembly of CA. Soluble HIV-1 CA can be triggered to assemble into tubes similar in diameter and morphology to intact cores by dilution into high-ionic-strength buffer. The kinetics of assembly can be followed by monitoring the increase in turbidity using a spectrophotometer. (Li et al., 2009, J. Virol. 83(21):10951-62; Tian et al., 2009, Bioorg. Med. Chem. Lett 19:2162-67). The assembly assay was performed in the presence of CMPD-E or DMSO alone. As shown in FIG. 13, compound CMPD-E prevented assembly of CA in vitro. Taken together, this body of data supports the docking model, in which CMPD-E binds at the interface of the two CA protomers and blocks hexamerization mediated by NTD-NTD interactions (FIG. 12A) (Mobley et al., 2009, Structure 17:489-98; Ohtaka et al., 2005, Prog. Biophys. Mol. Biol. 88:193-208).

### Example 5

### Mutation of Residues within the NTD of CA Reduces the Binding of CMPD-E

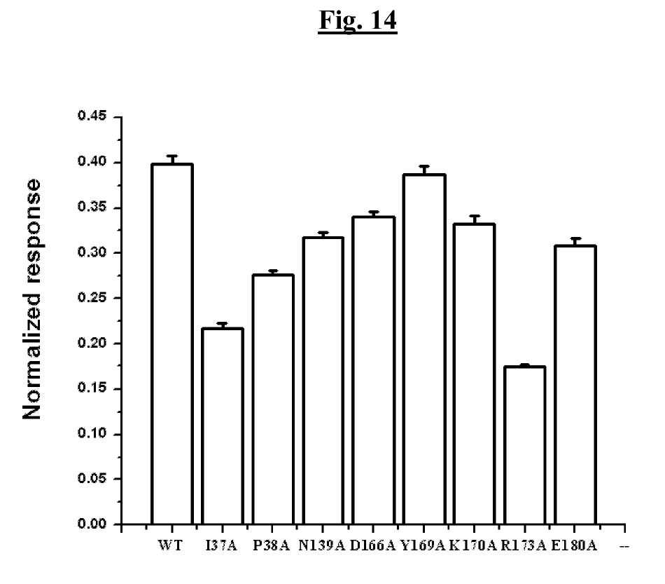

The combined results from SPR and ITC studies indicated that compound CMPD-E binds to HIV-1 CA with a 2:1 stoichiometry. This is consistent with results obtained from docking studies using CMPD-E that inferred that CMPD-E could potentially interact with both upper and lower regions of the NTD. Therefore, to further investigate the potential binding site(s) of CMPD-E on CA, mutations were created in the HIV-1 CA protein based on the docking models. Residues 137, P38, N139, D166, Y169, K170, R173 and E180 were mutated to alanine and their effect on the binding of a single concentration (27.5 μM) CMPD-E as compared to wild-type CA was assessed using SPR. From this analysis, residues 137, P38, N139 and R173 reduced the binding of CMPD-E as compared to wild-type CA to varying degrees, with residues 137 and R173 having the most pronounced effect (>2-fold reduction; FIG. 14). In contrast, PF74 localizes to an opposite pocket situated on the NTD of the CA protein, that is formed by helices 3, 4, 5 and 7. The binding pocket for PF74 as determined by co-crystallization studies involves residues Asn-53, Leu-56, Val-59, Gln-63, Met-66, Gln-67, Leu-69, Lys-70, Ile-73, Ala-105, Thr-107, Tyr-130 (Blair et al., 2010, PLoS Pathog 6:e1001220). The results described herein demonstrate that the binding of CMPD-E to HIV-1 CA is dependent on interactions with residues within the NTD and points to a novel site(s) of interaction than previously discovered CA inhibitors (FIG. 12B).

TABLE 1
Antiviral spectrum of CMPD-E.

| | IC₅₀ | TC₅₀ | Antiviral index |
|---|---|---|---|
| Virus strain* | (μM) | (μM) | (TC₅₀/IC₅₀) |
| DENV (serotypes 1-4) | >100 | >100 | NA |
| RSV | >100 | | NA |
| YFV | >100 | | NA |
| JEV | >100 | | NA |
| H1N1 | >100 | | NA |
| Vaccinia | >100 | | NA |
| Chikungunya | >100 | | NA |

DENV = Dengue virus;
RSV = respiratory syncytial virus;
H1N1 = influenza strain A/PR/8/34 (H1N1);
YFV = yellow fever virus 17D vaccine strain;
JEV = Japanese encephalitis virus 14-14-2.
NA = not applicable.

### Example 6

### CMPD-E is a Specific Inhibitor of HIV-1 Replication

The antiviral spectrum of CMPD-E was evaluated. To achieve this, CMPD-E was evaluated in CPE assays against a panel of viruses from different classes (Table 1). CMPD-E was evaluated against this panel of viruses up to a high-test concentration of 100 μM and displayed no inhibitory effects on the replication of Dengue serotypes 1-4, influenza H1N1, respiratory syncytial virus, yellow fever, Japanese encephalitis, Vaccinia, or Chikungunya viruses. Therefore, CMPD-E appears to be specific for HIV-1.

### Example 7

### CMPD-E Displays Broad Antiviral Activity Against Multiple Subtypes of HIV-1

A key issue in the development of novel HIV drugs is their ability to inhibit the replication of genetically diverse isolates, especially those isolates from the most globally prevalent subtypes, A, B, and C. Therefore, the antiviral efficacy of CMPD-E was evaluated in a standardized PBMC-based anti-HIV-1 assay with a panel of HIV-1 clinical isolates and laboratory strains from different geographic locations that included HIV-1 group M subtypes A, B, C, D, E, F, and G, as well as HIV-1 group 0 (Table 2). The panel included CCR5-tropic (R5), CXCR4-tropic (X4), and dual-tropic (R5X4) viruses.

CMPD-E inhibited the replication of viruses from all group M subtypes (A, B, C, and D, E, F and G), and also the group 0 isolate. Homology modeling of the available sequences of the isolates used in this study demonstrated a structural homology between the isolates of between 85 and 93% with reference to the crystal structure. Consistent with the antiviral analysis, CMPD-E docked with nearly similar profiles to all the isolates, albeit with slightly better scores for the Group 0 isolate.

TABLE 2
Therapeutic spectrum of I-XW-053

| | | | Antiviral index |
|---|---|---|---|
| Virus strain* | IC₅₀ (μM) | TC₅₀ (μM) | (TC₅₀/IC₅₀) |
| 92UG031 | 93.6 | >100 | >1.07 |
| 89BZ167 | 92.9 | | >1.08 |
| 92BR030 | 68.7 | | >1.46 |
| 92BR025 | 51.0 | | >1.96 |
| 93IN101 | 48.4 | | >2.07 |
| 92UG024 | 51.8 | | >1.93 |
| CMU08 | 100 | | >1 |
| 93BR020 | 65.6 | | >1.53 |
| G3 | 9.03 | | >11.1 |
| BCF02 | 71.3 | | >1.4 |

92UG031 = HIV-1 subtype A clinical isolate;
89BZ167 = HIV-1 subtype B clinical isolate
92BR030 = HIV-1 subtype B clinical isolate;
92BR025 = HIV-1 subtype C clinical isolate;
93IN101 = HIV-1 subtype C clinical isolate;
92UG024 = HIV-1 subtype D clinical isolate;
CMU08 = HIV-1 subtype E clinical isolate;
93BR020 = HIV-1 subtype F clinical isolate;
G3 = HIV-1 subtype G clinical isolate;
BCF02 = HIV-1 group O clinical isolate

### Example 8

### Single-Round Infection Assay

A single-round infection assay was used in order to determine whether the compounds identified by the HSB method affected early events (such as uncoating) or late events (such as assembly) or both. Details of the experimental procedure have been published (Rossi et al., Retrovirology 2008, 5:89; Cocklin et al., J. Virol. 2007, 81(7):3645-3648; Martin-Garcia et al., 2006, Virology 2006, 346(1):169-179; Martin-Garcia et al., 2005, J. Virol. 2005, 79(11):6703-6713).

Effects on assembly were identified by incubating the viral producer cells in the presence of the compound. Supernatants containing virus (that encodes for firefly luciferase as a reporter gene) were then diluted 10-fold and used to infect the target cells (U87 CD4-CXCR4). Compound-induced aberrant assembly was then manifested as a decrease in infectivity of the target cells, as compared to those infected with virus from untreated cells. Similarly, uncoating effects could be determined by producing virus in the absence of compound, then infecting target cells in the presence of compounds (U87 CD4-CXCR4).

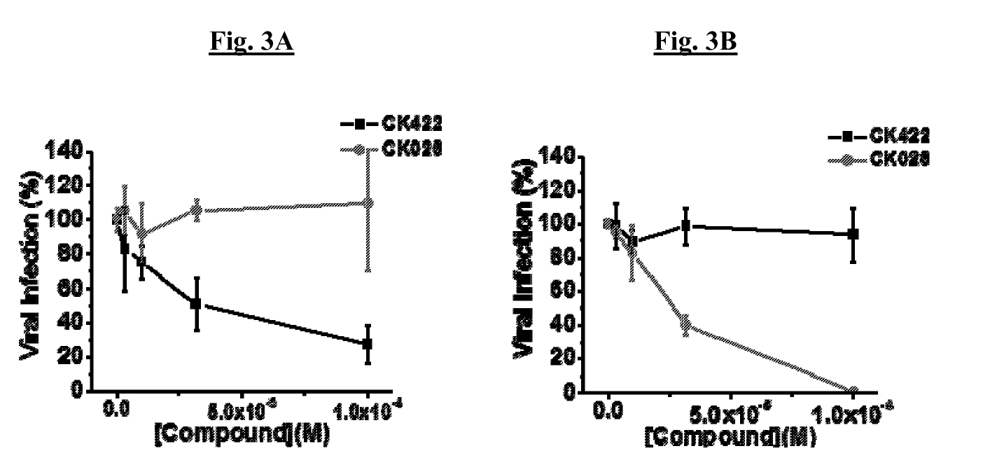

Screening of ten of the top-ranked commercially available molecules identified using the HSB method allowed the identification of CMPD-A and CMPD-D as compounds with significant antiretroviral activity against HIV-1 (FIG. 3). Moreover, these compounds appeared to be working at different stages of the viral life-cycle—one at an early post-entry event (CMPD-A) and the other at an assembly or post-integration event in HIV-1 replication (CMPD-D). The potential cytotoxicity of the compounds either on the producer cells (293T) or on the target cells (U87 CD4-CXCR4) was evaluated by measuring the release of the cellular enzyme lactate dehydrogenase (LDH) into the culture supernatants. Neither CMPD-A nor CMPD-D promoted LDH release from either treated producer or treated target cells in significantly higher amounts than those observed in the corresponding untreated cells (data not shown). The IC₅₀ value for compound CMPD-A against HIV-1 was determined as 33.6±0.31 μM.

Comparison of the MACCS (Molecular ACCess System) key fingerprints of compound CMPD-A with those of 115 HIV-1 inhibitors selected from PubChem demonstrated its novelty (data not shown). The purity and chemical composition of compound CMPD-A were confirmed by LCMS (data not shown). This process of demonstration of antiviral activity followed by chemical analysis may be performed on all commercially available compounds identified from the HSB study, after which the compounds may be resynthesized and retested. This procedure helps identify a battery of lead molecules from the commercially available compounds identified from the HSB screen that are suitable for optimization through iterative use of the HSB technique and state-of-the-art medicinal chemistry methods. This may result in second-generation therapeutics for HIV-1 intervention with improved potency and pharmacokinetic properties.

### Example 9

### Dengue Virus

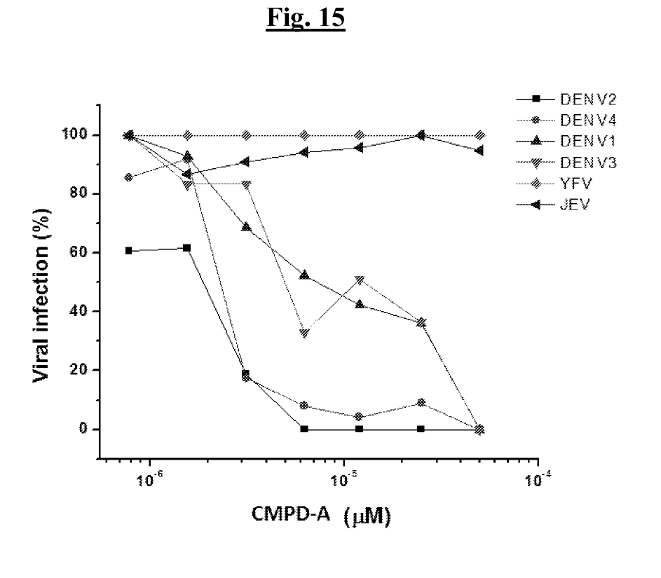

During the evaluation of CMPD-A, it was noted to have activity against DENV-2. Subsequent studies demonstrated that the compound inhibits all DENV serotypes 1-4 (FIG. 15) with an average half-maximal inhibitory concentration of 4.95 (±3.6) μM. CMPD-A displayed no cellular toxicity against the Vero cells over the concentration range tested. Independently, CMPD-A was also tested against DENV2 in a focus reduction assay using BHK cells. In this assay, the average IC₅₀ was found to be 1.25 (±1.1) μM, whereas the BHK cells were more sensitive to the compound with a CC₅₀ of 35 μM by MTT assay. The anti-HIV compound CMPD-E retains none of the anti-DENV activity of CMPD-A. As such, defining the molecular target and the active pharmacophore of this novel compound may reveal a hitherto unexplored new path in anti-DENV inhibitor development.

### Example 10

### West Nile Virus

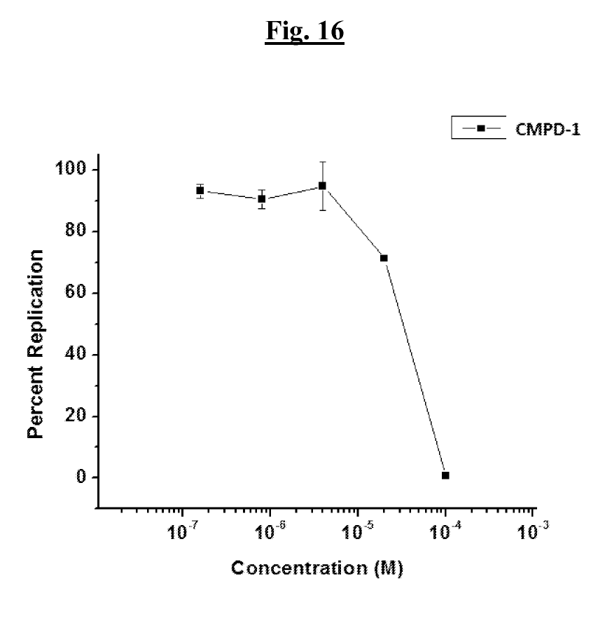

The activity of CMPD-A against a high-priority flaviviral pathogen, West Nile virus (WNV), was evaluated. The compound was tested in a West Nile Virus, virus-like particle (VLP) assay, which is a 24 hr assay on Vero cells using a firefly luciferase reporter to measure viral replication activity. In these tests IC₅₀ values of 30 (±13) μM were obtained, with no toxicity at 100 μM using either both MTT or a renilla luciferase cellular assay (FIG. 16). This result demonstrates specificity of the compound to WNV.

### Example 11

### Respiratory Syncytial Virus Inhibitor

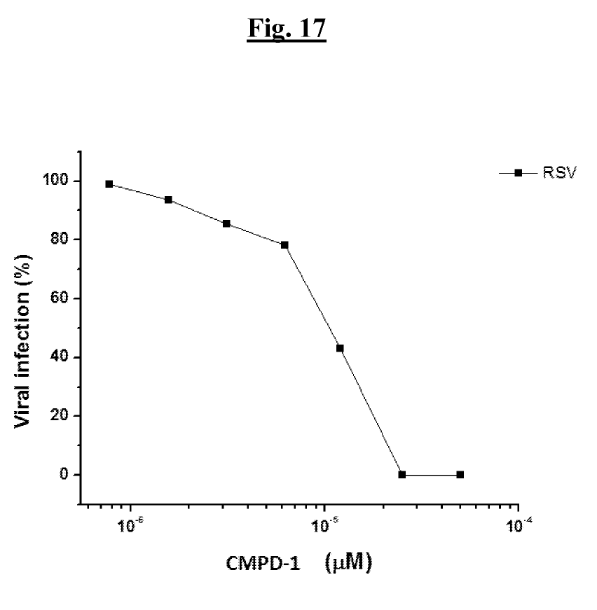

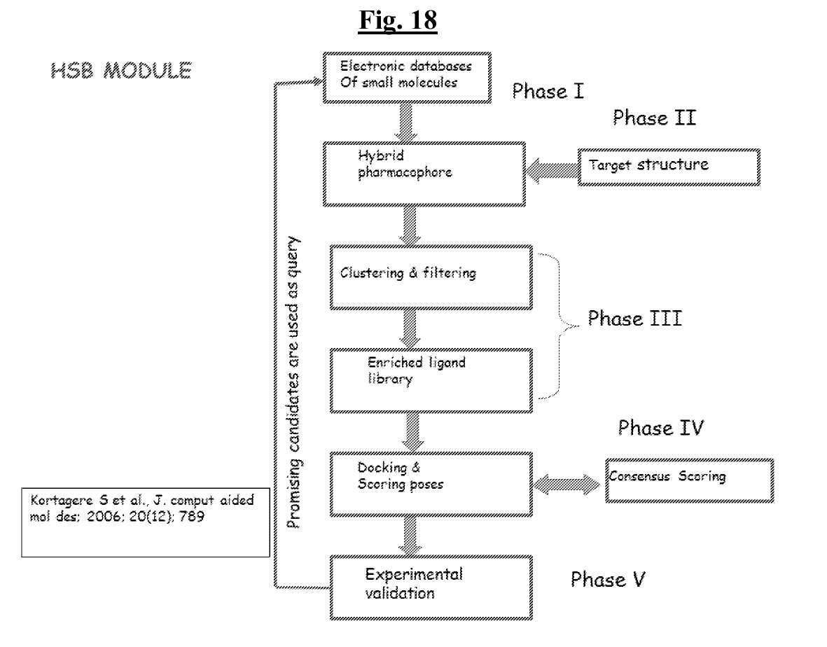

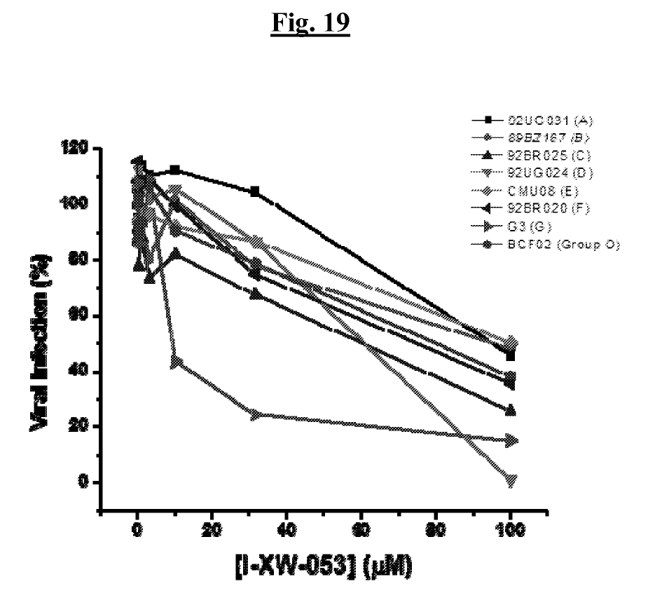

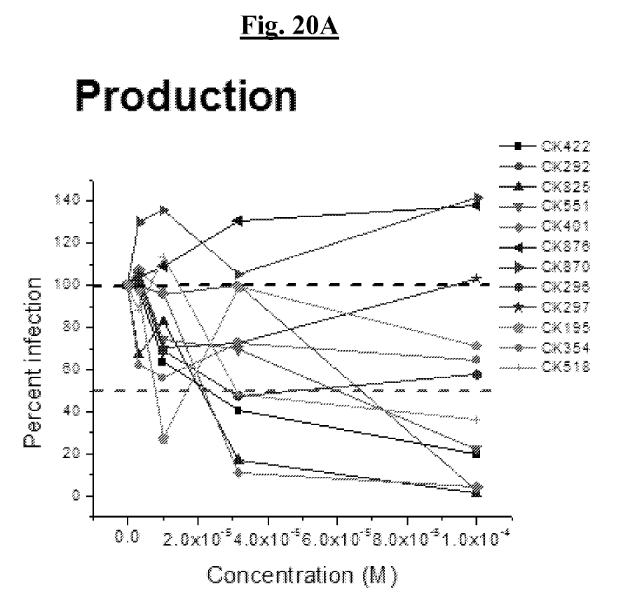

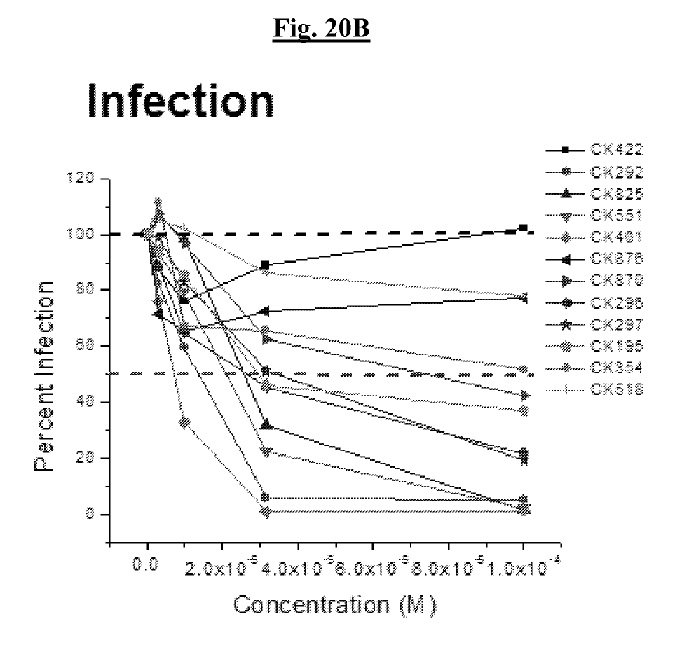

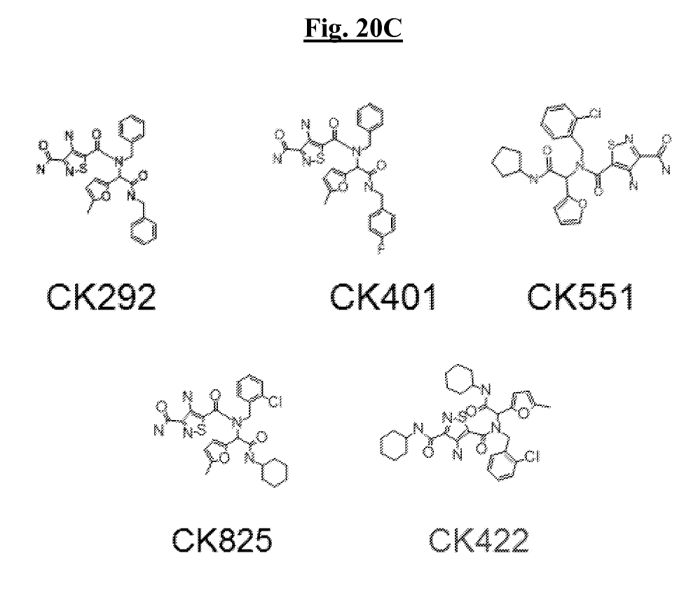

Studies assessing the antiviral spectrum of CMPD-A demonstrated that in addition to antiflaviviral activity the compound was active against RSV (FIG. 17). As such, defining the molecular target and the active pharmacophore of this novel compound may reveal a hitherto unexplored new path in anti-RSV inhibitor development.

The HIV-1 CA protein plays essential roles in both the early and late stages of viral replication and has recently emerged as an attractive target for drug discovery and development. The hybrid-structure based (HSB) method was herein used to identify small molecules that bind to the capsid NTD-NTD hexamerization interface and that are capable of disrupting HIV-1 replication at an early pre-integration event. Using the HSB methodology, 900 hits were obtained by pharmacophore-based screening and filtering of an over 3 million compound database. Of these 900 hits, 300 molecules belonging to different chemical cores (identified using principal component analysis on molecular descriptors derived from MOE) were subjected to further docking and scoring. Finally, the best ranked complexes were visually inspected for their potential to interact with CA but also to effectively disrupt the interaction of CA monomers with each other. Antiviral testing of the top ranked 25 compounds using a single round infection assay, identified 4,4′-[dibenzo[b,d]furan-2,8-diylbis(5-phenyl-1H-imidazole-4,2-diyl)]dibenzoic acid (CMPD-A) as a CA-targeted compound with the ability to disrupt HIV-1 replication at a post-entry, pre-integration event. This compound retained the ability to inhibit fully infectious HIV-1_{IIIB} replicating in the P4-R5 MAGI cells but was unable to inhibit the primary isolate HIV-1_{92BR030} replicating in PBMCs. This lack of efficacy in the PBMC assay was probably due to poor permeability in that system due to the unfavorable physical-chemical properties and large size of the compound. Therefore, the size of the compound was reduced in an attempt to improve its physical-chemical properties, while retaining its antiviral activity.

Based upon the docking pose of CMPD-A to HIV-1 CA, three analogues were synthesized; CMPD-B, CMPD-C, and CMPD-E. These analogues were subsequently assessed in the single-round infection assay yielding the finding that CMPD-E, the smallest of the analogues, retained all of the inhibitory capability of the parental compound. CMPD-E was submitted to secondary screening using primary isolates replicating in PBMCs. Unlike the parental CMPD-A, CMPD-E could inhibit the HIV-1_{92BR030} replicating in PBMCs. The combined use of SPR and ITC demonstrated specific interaction of CMPD-E with HIV-1 capsid and implicated residues in the NTD as being critical for this interaction. Using an in vitro CA assembly assay, it was determined that CMPD-E functioned by preventing the assembly of the capsid, consistent with the proposed mechanism of action as indicated by docking models. Moreover, compound CMPD-E demonstrated little or no cytotoxicity over the concentration range tested (up to 100 μM); is HIV-1 specific (showing no inhibition of a panel of nonretroviral DNA and RNA viruses; Table 1); and possesses broad-spectrum anti-HIV activity (Table 2).

Mutational analysis of potential CMPD-E-interacting residues on CA indicates that residues in the NTD and, more specifically, in the NTD-NTD interfacing region, are required for interaction of this compound with its target. This site includes residue R173, which is completely conserved across all HIV-1 strains. This binding region is in stark contrast to the binding sites of the other previously discovered CA inhibitors. CAP-1 was demonstrated by structural analyses to bind into a hidden pocket adjacent to the NTD-CTD interface and to prevent assembly by altering the local geometry required to make NTD-CTD interactions within the hexamer (Kelly et al., 2007, J. Mol. Biol. 373:355-66; Pornillos et al., 2009, Cell 137:1282-92). This CAP-1 binding site has subsequently been targeted by other improved CAP-1 derivatives (Jin et al., 2010, Bioorg. Med. Chem. 18:2135-40; Tian et al., 2009, Bioorg. & Med. Chem. Lett. 19:2162-67) and has also been found to be the binding site for new, more efficacious molecules (Titolo et al., 2010, 17th Conference on Retroviruses and Opportunistic Infections, San Francisco). Another region of CA that has been exploited is a conserved hydrophobic cleft within the CTD. This area, initially identified as the site of interaction of the CA-I peptide, has recently been successfully targeted using small molecules (Curreli et al., 2011, Bioorg. Med. Chem. 19:77-90).

The data disclosed herein suggest that the CMPD-E-binding site is distinct from that of PF74 (Blair et al., 2010, PLoS Pathog 6:e1001220) and imply a novel mechanism in which hexamerization is physically blocked by interaction of the compound with CA. Further studies are underway to determine the precise mechanism of action of CMPD-E and to define the binding site of the compound on HIV-1 CA. The compounds of the invention exhibit broad-spectrum anti-HIV activity, further highlighting the HIV-1 CA protein as a viable viral target with significant therapeutic potential.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A composition comprising a compound of Formula (III): 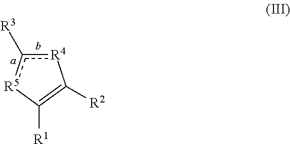
wherein in Formula (III):
R¹, R² and R³ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl, and
R⁴ and R⁵ are such that:
(i) if ‘a’ is a double bond and ‘b’ is a single bond, then R⁵ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or
(ii) if ‘a’ is a single bond and ‘b’ is a double bond, then R⁵ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂;
or a salt thereof.

2. The composition of claim 1, wherein in Formula (III) R⁴ and R⁵ are such that:
(i) if ‘a’ is a double bond and ‘b’ is a single bond, then R⁵ is N or CH, and R⁴ is NH or N-alkyl, or
(ii) if ‘a’ is a single bond and ‘b’ is a double bond, then R⁵ is NH or N-alkyl, and R⁴ is N or CH.

3. The composition of claim 2, wherein in Formula (III) R⁴ and R⁵ are such that:
(i) if ‘a’ is a double bond and ‘b’ is a single bond, then R⁵ is N, and R⁴ is NH or N-alkyl, or
(ii) if ‘a’ is a single bond and ‘b’ is a double bond, then R⁵ is NH or N-alkyl, and R⁴ is N.

4. The composition of claim 1, wherein said compound is 4-(4,5-diphenyl-1H-imidazol-2-yl)benzoic acid (CMPD-E) or a salt thereof.

5. The composition of claim 1, further comprising a pharmaceutically acceptable carrier.

6. A composition comprising a compound of Formula (Ib): 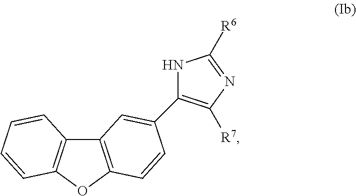
wherein in Formula (Ib) R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a salt thereof.

7. The composition of claim 6, wherein said compound is 4-(5-(dibenzo[b,d]furan-2-yl)-4-phenyl-1H-imidazol-2-yl)benzoic acid (CMPD-C) or a salt thereof.

8. The composition of claim 6, further comprising a pharmaceutically acceptable carrier.

9. A method of inhibiting, suppressing or preventing an HIV-1 infection in a subject in need thereof, said method comprising administering to said subject a composition comprising a therapeutically effective amount of at least one compound selected from the group consisting of:
(a) a compound of Formula (I): 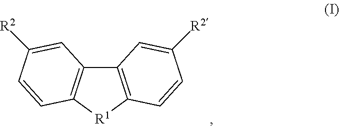
wherein in Formula (I):
R¹ is O, S, NH, N-alkyl, N—C(O)NH₂, N—CH₂C(O)NH₂, N—CH₂CH₂C(O)NH₂, CH₂, CH-alkyl, CH-OMe, CH-OEt, CH—C(O)NH₂, CH—CH₂C(O)NH₂, or CH—CH₂CH₂C(O)NH₂;
R² and R^{2′} are independently H or 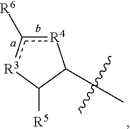
wherein,
(i) if ‘a’ is a double bond and ‘b’ is a single bond, then R³ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or
(ii) if ‘a’ is a single bond and ‘b’ is a double bond, then R³ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂;
with the proviso that if R² is H then R^{2′} is not H; and
R⁵ and R⁶ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl;
(b) a compound of Formula (II): 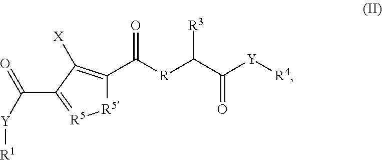
wherein:
R is NR₂, CHR₂, O or S;
R¹, R², R³ and R⁴ are independently H, alkyl, substituted alkyl, cycloalkyl, substituted cycloalkyl, aryl, substituted aryl, benzyl, substituted benzyl, heteroaryl, or substituted heteroaryl;
R⁵ is N or CH;
R^{5′} is CH₂, NH, S or O;
X is —NH₂, —NHR¹, —NR¹R², —OH, cyano, alkyl, alkoxy, halogen, sulfonamide, aryl, substituted aryl, heteroaryl or substituted heteroaryl; and,
each occurrence of Y is independently NH, NR¹, O, CH₂, CHR¹ or CR¹R²;
(c) a compound of Formula (III): 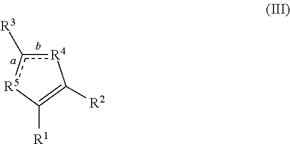
wherein in Formula (III):
R¹, R² and R³ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl,
R⁴ and R⁵ are such that:
(i) if ‘a’ is a double bond and ‘b’ is a single bond, then R⁵ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or
(ii) if ‘a’ is a single bond and ‘b’ is a double bond, then R⁵ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂;
a mixture thereof and a pharmaceutically acceptable salt thereof.

10. The method of claim 9, wherein said compound of Formula (I) is a compound of Formula (Ia): 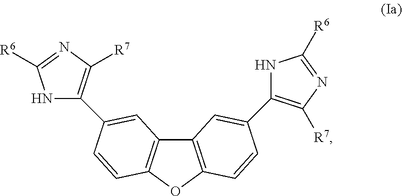
wherein in Formula (Ia):
R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

11. The method of claim 9, wherein said compound of Formula (I) is a compound of Formula (Ib): 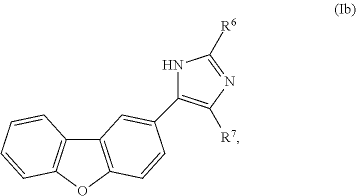
wherein in Formula (Ib):
R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

12. The method of claim 9, wherein in said compound of Formula (III) R⁴ and R⁵ are such that:
(i) if ‘a’ is a double bond and ‘b’ is a single bond, then R⁵ is N, and R⁴ is NH or N-alkyl, or
(ii) if ‘a’ is a single bond and ‘b’ is a double bond, then R⁵ is NH or N-alkyl, and R⁴ is N.

13. The method of claim 9, wherein said compound is selected from the group consisting of 4,4′-(5,5′-(dibenzo[b,d]furan-2,8-diyl)bis(4-phenyl-1H-imidazole-5,2-diyl))dibenzoic acid (CMPD-A), dimethyl 4,4′-(5,5′-(dibenzo[b,d]furan-2,8-diyl)bis(4-phenyl-1H-imidazole-5,2-diyl))dibenzoate (CMPD-B), 4-(5-(dibenzo[b,d]furan-2-yl)-4-phenyl-1H-imidazol-2-yl)benzoic acid (CMPD-C), 4-amino-N⁵-[(2-chlorophenyl)methyl]-N³-cyclohexyl-N⁵-[2-(cyclohexylamino)-1-(5-methylfuran-2-yl)-2-oxoethyl]-1,2-thiazole-3,5-dicarboxamide (CMPD-D), 4-(4,5-diphenyl-1H-imidazol-2-yl)benzoic acid (CMPD-E), 4-amino-N5-benzyl-N5-(2-(benzylamino)-1-(5-methylfuran-2-yl)-2-oxoethyl)isothiazole-3,5-dicarboxamide (CMPD-G), 4-amino-N5-benzyl-N5-(2-((4-fluorobenzyl)amino)-1-(5-methylfuran-2-yl)-2-oxoethyl) isothiazole-3,5-dicarboxamide (CMPD-H), 4-amino-N5-(2-chlorobenzyl)-N5-(2-(cyclopentylamino)-1-(furan-2-yl)-2-oxoethyl)isothiazole-3,5-dicarboxamide (CMPD-J), 4-amino-N5-(2-chlorobenzyl)-N5-(2-(cyclohexylamino)-1-(5-methyl-furan-2-yl)-2-oxoethyl)isothiazole-3,5-dicarboxamide (CMPD-K), a mixture thereof, and a salt thereof.

14. The method of claim 9, wherein said composition further comprises one or more anti-HIV drugs.

15. The method of claim 14, wherein said one or more anti-HIV drugs are selected from the group consisting of HIV combination drugs, entry and fusion inhibitors, integrase inhibitors, non-nucleoside reverse transcriptase inhibitors, nucleoside reverse transcriptase inhibitors, and protease inhibitors.

16. The method of claim 9, wherein said subject is a mammal.

17. The method of claim 16, wherein said subject is human.

18. A method of inhibiting, suppressing or preventing a viral infection in a subject in need thereof, said method comprising administering to said subject a composition comprising a therapeutically effective amount of at least one compound of Formula (I): 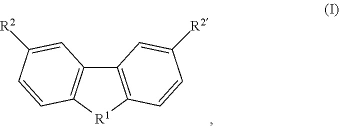
wherein in Formula (I):
R¹ is O, S, NH, N-alkyl, N—C(O)NH₂, N—CH₂C(O)NH₂, N—CH₂CH₂C(O)NH₂, CH₂, CH-alkyl, CH-OMe, CH-OEt, CH—C(O)NH₂, CH—CH₂C(O)NH₂, or CH—CH₂CH₂C(O)NH₂;
R² and R^{2′} are independently H or 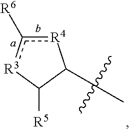
wherein,
(i) if ‘a’ is a double bond and ‘b’ is a single bond, then R³ is N, CH, C—OMe, C—OEt, C—C(O)NH₂, C—CH₂C(O)NH₂ or C—CH₂CH₂C(O)NH₂, and R⁴ is S, O, NH, N-alkyl, N—C(O)NH₂, N—CH₂C(O)NH₂ or N—CH₂CH₂C(O)NH₂, or
(ii) if ‘a’ is a single bond and ‘b’ is a double bond, then R³ is S, O, NH, N-alkyl, N—C(O)NH₂, N—CH₂C(O)NH₂ or N—CH₂CH₂C(O)NH₂, and R⁴ is N, CH, C—OMe, C—OEt, C—C(O)NH₂, C—CH₂C(O)NH₂ or C—CH₂CH₂C(O)NH₂;
with the proviso that if R² is H then R^{2′} is not H; and
R⁵ and R⁶ are independently alkyl, halo alkyl, substituted alkyl, alkoxy, aryl, substituted aryl, —SO₂NH₂, —SO₂NH-alkyl, —SO₂NH-aryl, —SO₂NH-substituted aryl, heteroaryl, substituted heteroaryl, alkoxycarbonyl, alkylthio, nitromethyl, or 2-nitroethyl, or a salt thereof,
wherein said viral infection comprises dengue fever, dengue hemorrhagic fever, dengue shock syndrome, West Nile virus infection, or respiratory syncytial virus infection.

19. The method of claim 18, said compound of Formula (I) is a compound of Formula (Ia): 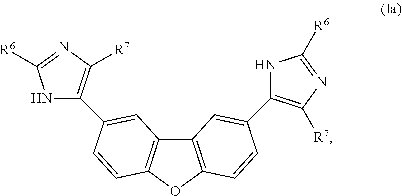
wherein in Formula (Ia):
R⁶ and R⁷ are independently alkyl, halo alkyl, substituted alkyl, alkoxy, aryl, substituted aryl, —SO₂NH₂, —SO₂NH-alkyl, —SO₂NH-substituted alkyl —SO₂NH-aryl, —SO₂NH-substituted aryl, heteroaryl, substituted heteroaryl, alkoxycarbonyl, alkylthio, nitromethyl, or 2-nitroethyl, or a pharmaceutically acceptable salt thereof.

20. The method of claim 19, wherein said compound is selected from the group consisting of 4,4′-(5,5′-(dibenzo[b,d]furan-2,8-diyl)bis(4-phenyl-1H-imidazole-5,2-diyl))dibenzoic acid (CMPD-A), dimethyl 4,4′-(5,5′-(dibenzo[b,d]furan-2,8-diyl)bis(4-phenyl-1H-imidazole-5,2-diyl))dibenzoate (CMPD-B), a mixture thereof, and a salt thereof.

21. The method of claim 18, wherein said subject is a mammal.

22. The method of claim 21, wherein said subject is human.


SEQUENCE LISTING (1 sequence)

SEQ ID NO: 1 - protein, 229 aa, Human immunodeficiency virus
PIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKAR